(19) United States
(12) Patent Application Publication
(10) Pub. No.: US 2026/0251642 A1
(43) Pub. Date: Aug. 27, 2026

(54) MOLECULAR TENSION SENSOR WITH NUCLEIC ACIDS AND HUH-TAGS

(71) Applicant: REGENTS OF THE UNIVERSITY OF MINNESOTA, Minneapolis, MN (US)

(72) Inventors: Wendy R. Gordon, St. Paul, MN (US); Matthew R. Pawlak, St. Paul, MN (US)

(21) Appl. No.: 18/992,445

(22) PCT Filed: Jul. 7, 2023
(86) PCT No.: PCT/US2023/027093

§ 371 Date: Jan. 8, 2025

Related U.S. Application Data
(60) Provisional application No. 63/359,612, filed on Jul. 8, 2022.

(51) Int. Cl. G01N 33/542; C07K 14/46; C12Q 1/6806; C12Q 1/6869

(52) U.S. Cl. CPC G01N 33/542; C07K 14/46; C12Q 1/6806; C12Q 1/6869; C07K 2319/80

(57) ABSTRACT

In one aspect the present disclosure relates to a molecular tension sensor including: a first nucleic acid strand nucleic acid; a second nucleic acid strand nucleic acid, wherein the second nucleic acid strand includes a region of at least five nucleotides that are complementary to a region of at least five nucleotides of the first nucleic acid strand; and an HUH-tag.

### CROSS-REFERENCE TO RELATED APPLICATION

This application claims the benefit of U.S. Provisional Patent Application No. 63/359,612 filed Jul. 8, 2022, which is incorporated herein by reference in its entirety.

### GOVERNMENT FUNDING

This invention was made with government support under GM119483 and CA009138 awarded by the National Institutes of Health. The government has certain rights in the invention.

### SEQUENCE LISTING

This application contains a Sequence Listing electronically submitted via Patent Center to the United States Patent and Trademark Office as an .xml file entitled “0110000699WO01.xml” having a size of 10 kilobytes and created on Jul. 5, 2023. The information contained in the Sequence Listing is incorporated by reference herein.

### SUMMARY

In one aspect the present disclosure relates to a molecular tension sensor including: a first nucleic acid strand nucleic acid; a second nucleic acid strand nucleic acid, wherein the second nucleic acid strand includes a region of at least five nucleotides that are complementary to a region of at least five nucleotides of the first nucleic acid strand; and an HUH-tag.

In one or more embodiments, the molecular tension sensor includes a ligand. The ligand and the HUH-tag may be or may be a part of a fusion protein. Additionally or alternatively, the ligand and the HUH-tag may be chemically ligated. In one or more embodiments, the ligand includes a nanobody, an antibody, or an scFv.

In one or more embodiments, the first nucleic acid strand is immobilized to a surface. The first nucleic acid strand may be immobilized to the surface by an affinity interaction, such as a biotin-avidin interaction. Additionally or alternatively, the first nucleic acid strand may be immobilized to the surface by adsorption.

In one or more embodiments, the first nucleic acid strand is 5 nucleotides to 80 nucleotides in length. In one or more embodiments, the second nucleic acid strand is 20 nucleotides to 100 nucleotides in length. The region of complementary nucleotides of the second nucleic acid strand may be 10 nucleotides to 30 nucleotides in length. The region of complementary nucleotides of the second nucleic acid strand may be at least 75%, at least 80%, at least 90%, or at least 95% complementary to a region of the first nucleic acid strand. The region of complementary nucleotides of the second nucleic acid strand may be 100% complementary to a region of the first nucleic acid strand.

In one or more embodiments, the second nucleic acid strand includes a fluorophore, for example, a small-molecule fluorophore, such as CY5, CY5.5, or CY3. In one or more embodiments, the first nucleic acid strand includes a fluorescence quencher. In one or more embodiments, the first nucleic acid strand, the second nucleic acid strand, or both includes modified nucleotides. For example, the first nucleic acid strand, the second nucleic acid strand, or both may include one or more peptide nucleic acid nucleotides. In one or more of these embodiments, the second nucleic acid strand includes a peptide nucleic acid and does not include DNA or RNA nucleotides.

In one or more embodiments, the second nucleic acid strand includes a barcode sequence. The barcode may include, for example, 5 nucleotides to 15 nucleotides.

In another aspect, the present disclosure relates to a plate for analysis of molecular tension in cultured cells including a molecular tension sensor described herein. The plate may be any suitable material, such as glass, or plastic, such as polystyrene. In one or more embodiments, the plate is coated with fibronectin, is silanized, or both. In one or more embodiments, the plate is coated with an affinity molecule, such as neutravidin or streptavidin.

In another aspect, the present disclosure relates to a kit for analysis of molecular tension in cultured cells. The kit may include a plate described herein, one or more buffers, and instructions for use. Additionally or alternatively, the kit may include a molecular tension sensor described herein, one or more buffers, and instructions for use.

In another aspect, the present disclosure relates to a method of preparing a molecular tension sensor, the method including providing a first nucleic acid strand immobilized to a substrate; providing a second nucleic acid strand, wherein the second nucleic acid strand includes a region of nucleotides complementary to the anchor stand and a region of nucleotides recognizable by an HUH-tag; hybridizing the second nucleic acid strand and the first nucleic acid strand, wherein hybridizing includes annealing the region of complementary nucleotides to form a hybridized assembly; and reacting the second nucleic acid strand with an HUH-tag, wherein the HUH-tag includes a ligand.

In another aspect, the present disclosure relates to a method of measuring molecular forces in a cell, the method including: providing a surface including a molecular tension sensor described herein; culturing a cell on the surface; measuring the molecular forces in the cell, wherein measuring includes measuring the presence of the molecular tension sensor; and interpreting the measurement of the presence of the molecular tension sensor as it relates to molecular forces. In one or more of these embodiments, measuring the presence of the molecular tension sensor includes measuring fluorescence. In one or more of these embodiments, fluorescence is measured using flow cytometry, high-resolution fluorescence microscopy, or both.

In one or more embodiments, a method further includes lysing the cell, extracting DNA from the cell, amplifying the DNA, and sequencing the DNA. In one or more of these embodiments, measuring the presence of the molecular tension sensor includes analyzing DNA sequencing results. The cell may be or may be related to a cancer cell, such as a glioma cell. Additionally or alternatively, the cell may be an immortalized cell or a primary cell.

### BRIEF DESCRIPTION OF THE DRAWINGS

FIG. 1. Schematic illustration of the use of rupture and deliver tension-gauge-tethers (RAD-TGTs) for high-throughput measurement of rupture of TGTs in the cell of interest and high-resolution surface imaging.

FIG. 2. Illustration of the design features of RAD-TGTs.

FIG. 3. Fluorescence data from CHO-K1 cells expressing a RAD-TGT. (A) Histograms of CY5 fluorescence from of CHO-K1 cells adhered to RAD-TGTs tethered to the HUH-tag wheat dwarf virus (WDV) or WDV-echistatin integrin ligand (WDV-Echi). (B) qTGT surface fluorescence imaging readout analogous to the histograms of FIG. 3A with CY5/Quencher swapped compared to FIG. 3A such that TGT rupture turns on fluorescence.

FIG. 4. Myosin II inhibitor para-amino blebbistatin (blebb) reduces rupture and delivery in CHO-K1 and U251 cells. (A) Representative histograms of vehicle (DMSO) and blebb treatment of cells adhered to WDV and WDV-Echi RAD-TGTs. Black solid and dashed lines denote median of DMSO and blebb treatments, respectively. (B) Superplots of biological replicates of median fluorescence fold changes upon drug treatments from FIG. 4A. Medians of the biological replicates are overlaid on dot plots of all of the data normalized to the corresponding WDV alone median.

FIG. 5. Flow cytometry data suggesting that Talin1 and CD44 CRISPR knockouts in U251 cells (TLN KO and CD44 KO cells, respectively) show reduced rupture and delivery of RAD-TGTs. (A) RAD-TGTs can be multiplexed to form 2D plots. Cells plated on surfaces containing CY5 labelled 12 piconewton (pN) RAD-TGTs and ALEXAFLUOR-488 labelled 54 pN RAD-TGTs all TGTs contained WDV-Echi. CHO-K1 cells (lighter grey) and U251 cells (darker grey) are shown as ALEXAFLUOR-488 versus CY5 dot plots. It should be noted that the ligand strand quencher was not present in this experiment, inclusion would decrease noise allowing for greater separation of populations. (B) Representative histograms of U251, U251-talinKO and U251-CD44KO cells plated on WDV and WDV-Echi RAD-TGTs, black line denotes median fluorescence. (C) Superplots of biological replicates of data shown in FIG. 5A as described in FIG. 4B.

FIG. 6. Rupture and delivery of RAD-TGTs with different ligand-receptor affinities. (A) representative histogram of U251 cells plated on WDV, WDV-FN, or WDV-Echi RAD-TGTs. (B) Superplots as described above. All statistics were performed using ANOVA of the medians of biological replicates.

FIG. 7. Data using DNA sequencing of barcoded RAD-TGTs as a measure of cellular tension. RAD-TGTs were conjugated to WDV, WDV-FN, and WDV-Echi, mixed 1:1:1 at ⅓ standard concentration and immobilized. U251 cells were plated, trypsinized and lysed. PCR of the barcodes of three replicates was performed and sent for forward and reverse next generation sequencing (NGS) and Sanger sequencing. Deconvolution analysis was performed as described in the methods. (A) Schematic of 12 pN RAD-TGT with barcode used for sequencing experiments. (B) Representative Sanger sequence (left) and analysis of the data (right). Mean % barcode for each replicate is denoted by the triangles, the center line within standard deviation bars is the total mean % barcode (SEQ ID NO:10). (C) Analysis of NGS data measuring the percentage of total reads each barcode composed, following the same convention as FIG. 7A. Each plot represents next-generation sequencing results of biological triplicates of U251 cells plated on an equimolar mixture of 12 pN RAD-TGTs with all three ligands present. **p=0.0077; ****p<0.0001.

FIG. 8. Gel electrophoresis showing WDV reacting with ssDNA and anchor strand-ligand strand duplex DNA. (A) Native gel electrophoresis showing WDV reacting with ssDNA and anchor strand-ligand strand duplex DNA. RAD-TGT components were reacted and analyzed in a non-denaturing polyacrylamide gel. The reaction for lane 1 contained WDV, the reaction for lane 2 contained the single-stranded DNA ligand from RAD-TGT and excess WDV, and the reaction for lane 3 contained anchor strand-ligand strand duplex DNA prepared by annealing the ligand and anchor strands from the RAD-TGT and excess WDV. Lane 1 contains a singular band representing unreacted WDV. Lanes 2 and 3 contain two bands, an upper band representing unreacted WDV and a lower WDV-DNA band. Because the DNA carries a negative charge, WDV migrates further on a native gel when bound to DNA. The band in lane 3 is shifted up relative to the band in lane 2 because the annealed duplex DNA increases the mass of the complex, resulting in slower migration. From this gel it was concluded that the WDV can react with anchor strand-ligand strand duplex DNA, as is present in RAD-TGTs. (B) An SDS-PAGE gel showing migration of the reactions. Lane 0 shows a molecular weight marker. Lane 1 shows unreacted WDV. Lane 2 shows WDV reacted with the ligand strand. Lane 3 shows WDV reacted with the annealed anchor strand-ligand strand duplex.

FIG. 9. Incubation time on RAD-TGTs increases fluorescent intensity. U251 cells were incubated on an echistatin conjugated RAD-TGT surface that also was coated with fibronectin. Cells were incubated for varying time points from 10 minutes to 120 minutes, once desired time was reached cells were dissociated and immediately analyzed via flow cytometry. This was performed with both 12 and 54 pN RAD-TGTs and a clear increase in signal over time was observed.

FIG. 10 shows the effect of seeded cell count on fluorescent intensity. A range of U251 cells (from 2000 to 20000) were plated on wells coated with echistatin-conjugated RAD-TGT and incubated for 90 minutes. Cells were then dissociated and the fluorescence intensity was analyzed with flow cytometry. Fluorescence intensity remained relatively constant regardless of cell seeding density.

FIG. 11. RAD-TGTs function with and without fibronectin. RAD-TGTs were plated on clean glass surfaces or on glass surfaces coated with fibronectin. U251 cells were plated on clean or fibronectin-coated surfaces with or without echistatin-conjugated RAD-TGT. TLN1 KO and CD44 KO U251 cells were also plated on clean or fibronectin-coated surfaces with echistatin-conjugated RAD-TGT. TLN1 KO and CD44 KO had decreased fluorescence signal relative to WT on all surfaces. Slight differences between the two conditions were attributed to cell behavior changing in a ligand dependent manner and competition between the RAD-TGTs and fibronectin for binding to the cell.

FIG. 12. RAD-TGTs retain function with TGTs of different rupture forces and on mixed TGT surfaces. Four different echistatin-conjugated RAD-TGTs conditions were compared to each other and a ligand-free negative control. WT U251 cells, TLN1 KO U251 cells, and CD44 KO U251 cells were independently plated on each echistatin-conjugated RAD-TGT condition. In all four conditions, half of the RAD-TGTs were fluorescently labeled with CY5 and half were unlabeled. Condition 1 (plots 2-4) only contained 12 pN RAD-TGTs, half of which were fluorescently labeled. Condition 2 (plots 5-7) contained fluorescently labeled 12 pN RAD-TGTs and unlabeled 54 pN RAD-TGTs, with only the 12 pN TGTs fluorescently labeled. Condition 3 (plots 8-10) contained fluorescently labeled 12 pN RAD-TGTs and fluorescently labeled 54 pN RAD-TGTs. Condition 4 (plots 11-13) contained 54 pN RAD-TGT, half of which were fluorescently labeled. Under all conditions, the WT U251 cells had a higher signal than either knockout. This aligns with the observations presented in FIG. 5C. The median values determined in FIG. 5C are represented by the horizontal black bars. These results indicate that RAD-TGTs allow for different forces to be tested individually and in conjunction with each other.

FIG. 13. Representative images of U251 cells grown on RAD-TGT surfaces. All U251 cells and variants were plated on surfaces containing echistatin conjugated RAD-TGTs and incubated for 90 minutes. Four conditions were tested, WT U251 cells, TLN1 KO U251 cells, CD44 KO U251 cells, and WT U251 cells treated with 50 μM para-amino-blebbistatin.

FIG. 14. Representative histograms of CY5 fluorescence intensity of U251 cells plated on polystyrene surfaces. Neutravidin-coated polystyrene plates were coated with echistatin-conjugated RAD-TGTs. A second set of neutravidin coated polystyrene plates were coated with WDV-conjugated (no ligand) RAD-TGTs. WT U251 cells were incubated on each surface, either with or without para-amino-blebbistatin treatment. TLN1 KO U251 cells were also incubated on each surface. The vertical line is representative of the median CY5 fluorescence intensity from cells plated on echistatin-conjugated RAD-TGTs. The distribution of events is broad relative to experiments on glass plates and a tail of high fluorescence intensity forms in the presence of histatin. These results are attributed to non-specific binding promoted by the readily adhering polystyrene causing uneven distribution of RAD-TGTs and fluorescently labeled oligos.

FIG. 15. Soluble RAD-TGT titration highlights differences in integrin composition between cell lines. Soluble RAD-TGTs composed of the echistatin conjugated to fluorescently labeled ligand strand annealed to the non-anchor strand was added into the wells at varying concentrations ranging from 0 to 0.89 M. The total picomolar (pmol) range of oligo was from 0 to 160 μmol. Thus, the standard RAD-TGT experiment in the main text contained 80 μmol oligo assuming all oligo properly adhered, thus this is representative of experimental conditions. From this it was concluded that U251 cells internalize the soluble RAD-TGT at a greater rate than the CHO-K1 cells,

FIG. 16. Fluorescent intensity and fold change of RAD-TGTs with different ligands. U251 cells were plated on RAD-TGT surfaces that were either conjugated to no ligand (WDV), a fibronectin derived ligand (FN), or echistatin (Echi). Fluorescence intensity was measured with flow cytometry (A). Median fluorescent value was measured for each condition and fold change was calculated for each ligand by dividing the ligand's median value by WDV's value (B). Echi conjugated RAD-TGTs had greater fluorescent signal than FN. This was attributed to the high affinity of echistatin for integrins.

FIG. 17. A comparison of RAD-TGTs conjugated to different ligands. CHO-K1 cells (left) and U251 cells (right) were plated on CY5-labeled RAD-TGTs conjugated to WDV, WDV-Fibronectin (FN), or WDV-Echi and treated with DMSO or para-amino-blebbistatin. Though the fold-change from WDV is smaller for the FN ligand than the Echi ligand, blebbistatin still reduces median fluorescence.

FIG. 18. A flow cytometry readout of multiple ligands in one well as done in sequencing experiments. U251 cells were plated in wells containing equimolar amounts of three unique RAD-TGTs one for each ligand (WDV, FN, or Echi) analogously to the sequencing experimental setup. Unlike the sequencing experiment, one RAD-TGT per well was fluorescently labeled, and three wells were prepared so that each well contained fluorescently labeled RAD-TGTs conjugated to one of the 3 ligands. Each well was then analyzed via flow cytometry and the recorded fluorescence was attributed to the labeled ligand.

FIG. 19. Conceptual schematic of the force-induced rupture and readout of RAD-TGTs.

FIG. 20. Brightfield imaging at 20× of adhesion assay of cells on RAD-TGT surfaces with either WDV, WDV-FN, or WDV-Echi ligand and 12 pN or 54 pN rupture force.

FIG. 21. Fluorescent imaging at 40× of qTGT fluorescent duplex rupture of cells plated on TGT surfaces of varying ligand and rupture force composition. Dotted lines denote cell borders.

FIG. 22. Flow cytometry results of cells plated on RAD-TGT surfaces with different ligand and rupture force composition

FIG. 23. Representative histogram showing the effect of para-amino-blebbistatin treatment on TGT rupture.

FIG. 24. Superplots of CY5 fluorescence of each cell from three biological replicates normalized to WDV median fluorescence with symbols for medians of biological replicates. Representative histograms turned 90° and scaled linearly for reference. Outlined histograms are WDV only, dotted lines are with para-amino-blebbistatin, and solid are with DMSO treatment as control.

FIG. 25. Superplots of the fold change of CY5 fluorescence intensity of individual U251 cells plated on 12 pN (top) or 54 pN (bottom) RAD-TGTs with either WDV, WDV-FN, or WDV-Echi. Triangles represent the median fold change of population per replicate; the horizontal line is at the median fold change for all cells analyzed. n=8 independent experiments for 12 pN; n=5 independent experiments for 54 pN. ***p=0.0002, ****p<0.0001.

FIG. 26. Brightfield and qTGT imaging of U251 cells plated on either 12 pN or 54 pN qTGTs with WDV-Echi ligand.

FIG. 27. RAD-TGTs detect CRISPR-KO of mechanosensing proteins in single and mixed cell populations. (A) Superplots of the fold change of CY5 fluorescence intensity of individual WT, TLN1 KO, or CD44 KO U251 cells on 12 pN (top) or 54 pN (bottom) RAD-TGTs with WDV-Echi. Symbols represent the median Cy5 fold change for each replicate experiment. Identical symbols indicate the samples were collected on the same day. The horizontal line is at the median fold change for all cells analyzed. For 12 pN experiments, n=8 (WT), 3 (CD44KO), or 6 (talinKO) and for 54 pN experiments n=5 (WT) and 3 (CD44KO and talinKO). A two-tailed paired t-test was used for statistics, *p=0.0360 (12 pN CD44KO), **p=0.0028 (12 pN talinKO), and *p=0.0322 (54 pN talinKO). Medians were paired based on the day each experiment was performed. (B) Histogram of a mixed population of U251 WT, Talin1, and CD44 KO (gray solid), overlaid with histograms of cell lines tested individually (outlines only). (C) Violin plots of individual populations, mixed population, and the sum of individual populations

FIG. 28. Schematic illustration of U251 WT and CD44 KO cells and accompanying histogram of the two cell types mixed together or individually plated. The composition of the population is determined by measuring the GFP signal on different applied gates of the CY5 histogram as CD44 KO also expresses GFP. The composition of the lowest 20%, highest 20%, and the entire population is displayed.

FIG. 29. Scatter plots of U251 and CHO-K1 cells plated on surfaces containing a 12 pN Cy5 labeled RAD-TGT and 54 pN A488 labeled RAD-TGT for both each cell individually and in a mixed population.

FIG. 30. RAD-TGT readout with or without quencher on bottom strand. U251 cells were incubated on 12 pN or 54 pN RAD-TGT coated surfaces. Each condition was performed with and without a quencher present on the bottom strand. Cy5 intensity was measured for each condition and associated fold change of CY5 fluorescence relative to WDV only was calculated and graphed. Horizontal lines represent the median and interquartile range. (A) Bulk Cy5 intensity measured for each condition. (B) Fold change with vs without quencher for each mechanosensor.

FIG. 31. Brightfield and wider view images corresponding to FIG. 21.

FIG. 32. Quantification of fluorescence intensity density per cell. A ROI corresponding to a cell was chosen in ImageJ, the intensity density was measured across the cell area. 4-10 cells were analyzed per condition. (A) Cells corresponding to those described in FIG. 21. (B) Cells corresponding to those described in FIG. 27B.

FIG. 33. Nuclease treatment of U251 cells. U251 cells were plated on Cy5-labeled RAD-TGTs conjugated to WDV-Echistatin. Following a 90-minute incubation cells were trypsinized and resuspended in a modified resuspension buffer that did not contain any EDTA (1% BSA in PBS). 2 mM MgCl₂ and 0.5 U/μL benzonase (Millipore, E1014) were added to the solution; for no nuclease treatment conditions benzonase buffer (50% glycerol containing 20 mM Tris HCl, pH 8.0, 2 mM MgCl₂, and 20 mM NaCl) was used in lieu of benzonase. Solutions were incubated at 37° C. for 10 minutes followed by addition of 10 mM EDTA to quench the reaction. Cells were then analyzed via flowcytometry as previously described. (A) A representative histogram of cells not treated with benzonase. (B) A representative histogram of cells treated with benzonase.

FIG. 34. Quantification of the fluorescence intensity shown in FIG. 33. Median Cy5 intensities of three biological replicates of WDV and WDV-Echi TGTs with and without benzonase.

FIG. 35. Incubation time on RAD-TGTs increases fluorescence intensity. U251 cells were incubated on echistatin conjugated 12 pN and 54 pN RAD-TGT surfaces. Cells were incubated for varying time points from 10 minutes to 120 minutes, once the desired time was reached, cells were dissociated and immediately analyzed via flow cytometry. The median fluorescence at each time point was normalized for the WDV alone median fluorescence. The graph shows two biological replicates, except at 90 minutes. The line represents the average fold change of the two experiments.

FIG. 36. Effect of seeded cell count on fluorescent intensity. A titration of U251 cells (from cell count 3750 to 30,000) were plated on WDV or WDV-Echi conjugated to 12 pN RAD-TGT wells and incubated for 90 minutes in 96-well glass bottom plates (MatTek Corp., Ashland, MA). Dots represent Cy5 fluorescence of each cell combining three biological replicates. Fold change relative to WDV MFI was calculated for each density. Symbols represent the median fold change of fluorescence per biological replicate. Horizontal line is at the median for each condition. Statistics were performed using a one-way ANOVA of the medians of three biological replicates. N.s p>0.05.

FIG. 37. RAD-TGTs Function with and without fibronectin present. 12 pN RAD-TGTs were plated on both clean glass surfaces and surfaces that were coated with 12.5 μg/ml fibronectin. U251 cells were plated on both surfaces and with either WDV or WDV-Echi RAD-TGTs and were treated with para-amino-blebbistatin or DMSO as a vehicle control. Dots represent Cy5 fluorescence of each cell combining all three biological replicates. Symbols represent the median fold change of fluorescence of WDV-Echi compared to WDV alone per biological replicate. Statistics were calculated by two tailed paired T-test of median fold change values. **p=0.0041 (+FN), 0.006 (−FN) The trend between cells on glass or fibronectin was preserved. Horizontal line is at the median for each condition.

FIG. 38. Representative histograms of U251 cells on polystyrene plates. U251 cells were incubated on an echistatin conjugated RAD-TGT surface as described in the main text but rather than glass plates neutravidin coated polystyrene plates (Thermo Fisher Scientific, Inc., Waltham MA) were used so no biotinylated-BSA or neutravidin was added to the plate. Surfaces contained either no ligand WDV conjugated RAD-TGTs or echistatin conjugated RAD-TGTs present. Wild type U251 cells were tested with and without para-amino-blebbistatin treatment and U251 TLN1 KO cells were also measured. The distribution of events is broad relative to experiments on glass plates and a distinct tail forms in the presence of echistatin. These results were attributed to non-specific binding promoted by the readily adhering polystyrene causing uneven distribution of RAD-TGTs and fluorescently labeled oligos.

FIG. 39. RAD-TGT Signal is Not Directly Related to Spread Area. Images of the cells before RAD-TGT readout were captured and the area (pixels) was then measured using FIJI. This was performed on both 12 pN and 54 pN RAD-TGTs using both WDV-Echi and WDV treated with a vehicle control (DMSO) or blebb. Each point represents median cell area per image, horizontal lines and accompanying error bars represent the mean and SEM, each circle represents an individual cell measured. At least eight cells were measured per WDV-echi or WDV-FN condition and at least three cells were measured per WDV only conditions.

FIG. 40. Brightfield images of U251 on different ligand-conjugated RAD-TGTs.

FIG. 41. Comparing percent positive versus median fluorescence methods of analyzing data for data presented in FIG. 25.

FIG. 42. Fold change Superplots quantifying median fluorescence intensity. The horizontal line is set at the median value of the replicates. The horizontal line is at the median fold change for all cells analyzed. n=8 independent experiments for 12 pN; n=5 independent experiments for pN. ***p=0.0002, ****p<0.0001, ns p>0.05

FIG. 43. Quantification of treated cells. (A) Blebbistatin treatment reduces RAD-TGT rupture by U251 cells. Fold change in CY5 fluorescence normalized to median fluorescence of WDV alone. Points are all fluorescent points from three biological replicates, while symbols are median of each data set. Statistics were calculated by one-way ANOVA of median fold change values of biological triplicates. ns p>0.05, *p=0.0151. Horizontal line is at the median for each condition. (B) Percent of U251 cells positive for fluorescence. A gate was drawn such that the negative control population is ~95% below the gate.

FIG. 44. U251 have little surface nuclease activity. Both U251 and CHO-K1 cells were plated onto 54 pN qTGT surfaces that also contained fibronectin to allow for proper adhesion. The positive controls contained WDV-Echi so that one may visualize the normal rupture intensity. The surface nuclease sensor was similar in design but only had WDV present so there was no ligand. Both brightfield (left) and fluorescent images (right) were captured to see overall shape of the cell and any gain of fluorescence from duplex dissociation from either force or nuclease activity.

FIG. 45. Treatment of U251 cells with surface nuclease sensors. (A) Schematic diagram showing of how surface nuclease sensors function, gaining fluorescence if a nuclease is encountered without the need of a ligand. (B) Flow cytometry data at ⅓ Ligand Concentration that Corresponds to Sequencing Data. RAD-TGTs with unique barcodes were conjugated to WDV, WDV-FN, and WDV-Echi and plated in a 96-well plate. Cells were plated, dissociated, and analyzed by flow cytometry while a duplicate sample was sent for sequencing. Statistics were performed using a one-way ANOVA of all cells collected per condition. n.s. p>0.05, ****p<0.0001. Horizontal line is at the median for each condition.

FIG. 46. Representative Images of U251 Cells on RAD-TGT surface. All U251 cells and variants were plated on surfaces containing echistatin conjugated RAD-TGTs and allowed to incubate for 90 minutes. In total, four conditions are shown here, U251 WT, U251 TLN1 KO, U251 CD44 KO, and U251 treated with 50 μM para-amino-blebbistatin.

FIG. 47. Multiplex RAD-TGTs with inverted fluorophores. Scatter plots of U251 and CHO-K1 cells plated on surfaces containing a 12 pN A488 labelled RAD-TGT and 54 pN Cy5 labelled RAD-TGT for both each cell individually and in a mixed population. The fluorophores and TGTs are inverted compared to main FIG. 28.

FIG. 48. Gating strategies used for flow cytometry. (A) Population of cells was first identified by creating a forward vs side scatter Logicle plot. (B) Population identified in a was then then gated to isolate single cells only, this was done by graphing the forward scatter area by height and gating for cells that display a linear relationship. (C) Cells from (B) are then gated to remove any non-fluorescent data points. Cells were displayed as a histogram of the fluorescent intensity of the fluorophore of interest and any point with an intensity greater than 10 were collected.

### DETAILED DESCRIPTION OF ILLUSTRATIVE EMBODIMENTS

This disclosure describes a molecular tension sensor including a first nucleic acid strand, a second nucleic acid strand, an HUH-tag, and a ligand of interest. This disclosure additionally discloses methods making and using the same. This disclosure describes a plate including the molecular tension sensors described herein, a kit including the molecular tension sensors described herein, and methods of making the same.

Mechanical force has emerged as a regulator of cell behavior to drive diverse biological processes from cell migration and stem cell differentiation to discrimination among similar T-cell antigens. Moreover, dysregulated cellular tensions in disease often leads to distinct mechanical phenotypes in comparison to normal cells; breast cancer cells are stiffer, while metastatic breast cancer cells are more compliant that normal cells. These changes in mechanical phenotype have been linked to disease progression. Mechanosensing proteins in the cell sense physical stimuli in the microenvironment and convert them into a biological response via protein conformational changes. This process is known as mechanotransduction. Mechanosensing pathways have been the focus of recent drug development. Thus, identification of mechanosensing proteins that mediate disease-related mechanical phenotypes may lead to new therapeutic targets.

Cellular forces on the scale of piconewtons (pN) are typically quantified using methods such as traction force microscopy (TFM) and molecular tension sensors. Numerous variations of molecular tension sensors have been developed in the last decade or so, including genetically-encoded Förster resonance energy transfer (FRET) and bioluminescence resonance energy transfer (BRET) tension sensors as well as surface-immobilized peptide, DNA hairpin, and DNA duplex based tension sensors. Molecular tension sensors are based on applied force altering the conformation of a “molecular spring” component and an optical readout of the molecular change, such as a change in fluorescence or energy transfer. These sensors are typically read out using high-resolution fluorescence microscopy. However, the low throughput nature of reading out forces by high-resolution microscopy as well as complex preparation of elastic surfaces and functionalized DNA-based tension sensors have precluded studies of the mechanosome from entering the -omics era.

One recent development is DNA-based molecular tension sensors called Tension-Gauge-Tethers (TGTs), which provide an irreversible and threshold-based readout of tension. TGTs are formed of DNA duplexes in which a first nucleic acid strand is immobilized to a surface, referred to herein as the “anchor strand,” and a second nucleic acid strand is conjugated to a ligand that recognizes a cell surface receptor, referred to herein as the “ligand strand.” When cells are plated on top of TGTs, forces associated with adhesion and migration can mechanically rupture TGT anchor strand-ligand strand duplexes. A fluorophore or fluorophore quencher pair incorporated into the oligos allows readout of TGT rupture via fluorescence microscopy of the surface, where gain or loss of fluorescent signal is proportional to the total number of accumulated mechanical events over time. Moreover, the tension threshold of DNA duplexes is tunable according to GC content and/or anchoring point of the anchor strand to the surface, allowing measurement of multiplexed forces.

However, current versions of TGTs generally require high resolution imaging of the surface to readout duplex rupture or measuring cell-mediated forces using flow cytometry of silica beads presenting DNA tension probes. Current TGTs also require specialized glass surfaces and arduous chemical modifications of DNA to conjugate TGTs to ligands/surface, limiting their potential for high throughput assays and widespread use.

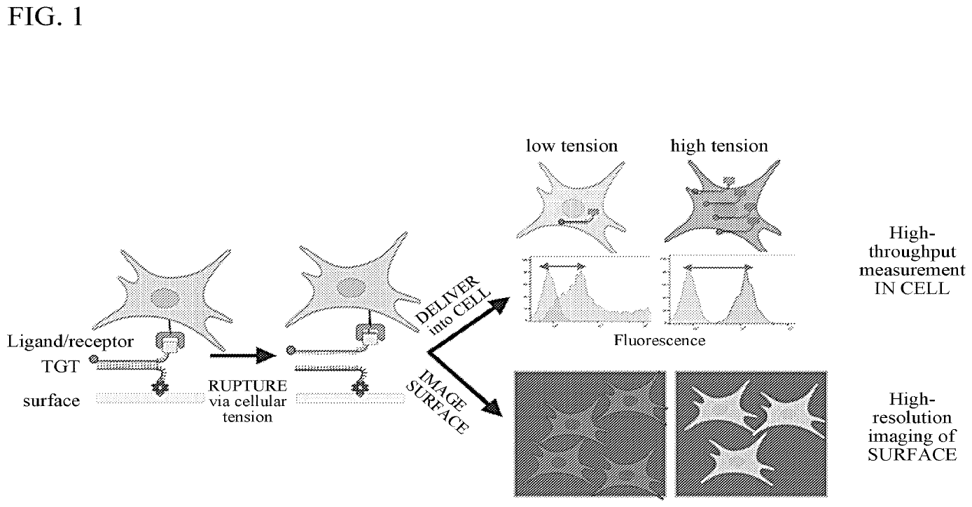

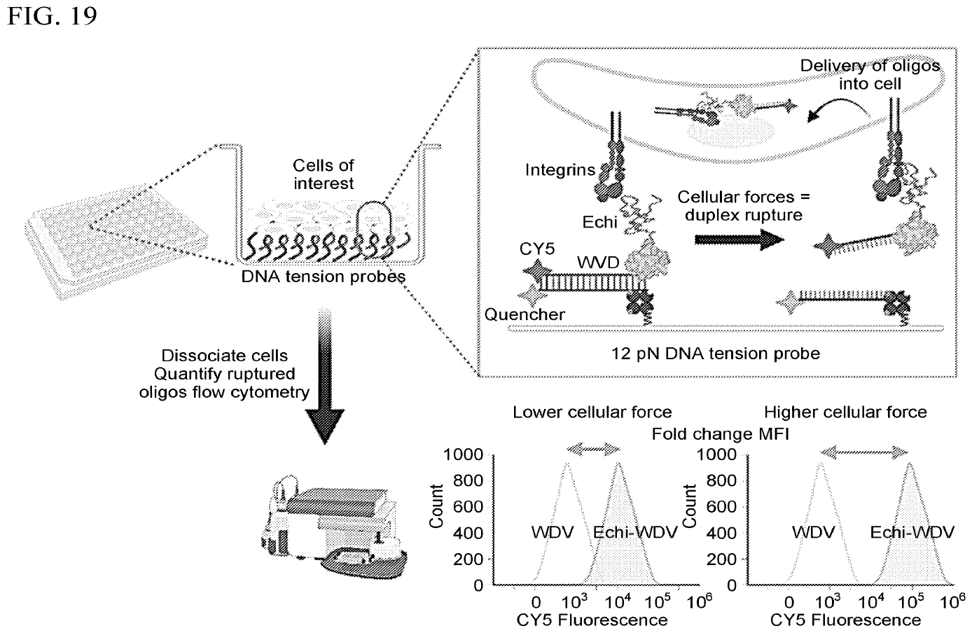

In one aspect, this disclosure describes “Rupture And Deliver” DNA duplex molecular tension sensors referred to herein as “RAD-TGTs”. RAD-TGTs may be modularly assembled for rapid testing of multiple ligands of interest. RAD-TGTs may be used to measure molecular tension using fluorescence microscopy in addition to high-throughput readouts, such as flow cytometry and DNA sequencing as shown in FIG. 1 and FIG. 19.

### Design of Molecular Tension Sensors

TGT designs typically include two strands of nucleic acid, typically DNA. A first nucleic acid strand is alternately referred to herein as an anchor strand. The anchor strand is typically an anchor strand that is immobilized on a surface. A TGT design also includes a second nucleic acid strand, referred to herein as a ligand strand. The ligand strand is typically conjugated to a ligand. The anchor strand includes a terminal biotin modification either at the 5′ or 3′ end that binds plated neutravidin. The anchor strand and the ligand strand include a complementary region, through which they form a double-stranded duplex. The ligand strand is attached to a ligand, which is bound by a cell. In this way, the TGT forms a connection between a cell and the surface on which it is plated. When the TGT is subjected to tension, the complementary region may rupture, either by unzipping in response to force of at least 12 pN or shearing in response to force of at least 54 pN. While TGTs provide a useful method for measuring cellular tension, their preparation can be cumbersome and time-consuming. Attachment of a ligand to the ligand strand may require complex conjugation chemistry and specialized reagents. Typically, the ligand strand of TGTs is chemically modified with a ligand such as integrin-binding cyclic-RGD using amino, thiol, or click chemistry, which require specialized equipment and knowledge to perform. Additionally, current TGT designs are usually read out by fluorescence microscopy, which may be time- and labor-intensive.

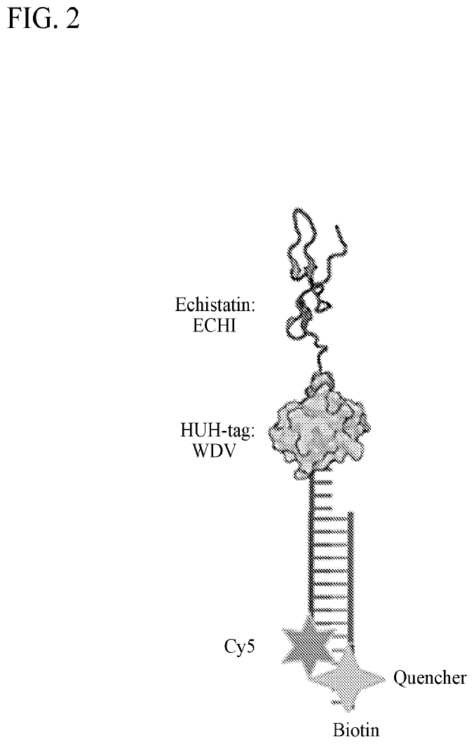

In one or more embodiments, a RAD-TGT is a molecular tension sensor including an anchor strand of nucleic acid, a ligand strand of nucleic acid, wherein the ligand strand has a region of at least five nucleotides that are complementary to a region of at least five nucleotides of the anchor strand, and an HUH-tag. RAD-TGTs are designed to enable modular attachment of different ligands to a preexisting anchor strand-ligand strand duplex. RAD-TGTs may be prepared simply with “off the shelf” oligos, leveraging covalent DNA-linking HUH-tag to attach desired recombinant protein ligands of interest to unmodified DNA. In this design, the ligand strand includes an HUH recognition sequence on one terminus, which may then be bound by an HUH-tag fused to a ligand of interest as shown in FIG. 2.

In one or more embodiments, the anchor strand of a molecular tension sensor includes a moiety configured to immobilize the anchor strand to a support. An immobilized anchor strand may or may not be covalently attached to a support. An immobilized anchor strand is typically attached to a surface with a strength sufficient to prevent disassociation in response to tension up to a predetermined strength. For example, an anchor strand may be immobilized to a support such that the anchor strand remains attached to the support when subjected to tension of at least 1 nanonewton (nN), such as at least 500 pN, at least 400 pN, at least 300 pN, at least 200 pN, at least 100 pN, at least 75 pN, or at least 50 pN.

In one or more embodiments, the anchor strand of a molecular tension sensor is immobilized to a support via a biotin-streptavidin interaction. The anchor strand may include a biotin moiety or a streptavidin moiety. In one or more embodiments, the anchor strand includes a biotin moiety on its 5′ end. For example, the anchor strand may include a biotin moiety attached to the terminal 5′ phosphate. In one or more embodiments, the anchor strand includes a biotin moiety on its 3′ end. For example, the anchor strand may include a biotin moiety attached to the terminal 3′ hydroxyl. Alternatively, the anchor strand may include a biotin moiety in a 5′ or a 3′ region, such as within the last five nucleotides of either the 3′ end or the 5′ end. In one or more embodiments, the ligand strand of a molecular tension sensor may be 20 to 100 nucleotides in length such as, for example, 30 to 80 nucleotides in length, or 45 nucleotides in length. In one or more embodiments, the anchor strand of a molecular tension sensor may be 5 to 80 nucleotides in length such as, for example, 15 to 60 nucleotides in length, or 20 nucleotides in length. The ligand strand may include a region of nucleotides that are complementary to the anchor strand to facilitate hybridization of the two strands. In one or more embodiments, the complementary region may be 5 to 30 nucleotides in length, 10 to 20 nucleotides in length, or 15 nucleotides in length. In one or more embodiments, the complementary region may include at most ten mismatches, at most five mismatches, at most three mismatches, or at most one mismatch between the two strands. In one or more embodiments, the complementary region may include no mismatches.

HUH-tags are a class of small (<40 kDa) DNA-binding proteins derived from HUH endonucleases. HUH-tags form robust, sequence-specific phospho-tyrosine covalent bonds with a short non-nucleotide sequence of ssDNA within minutes under physiologic conditions. HUH-tags may be expressed as fusion proteins with any compatible ligand of interest. Thus, the ligand strand of a RAD-TGT does not require chemical modification to facilitate connection to the ligand as in other TGT designs, but simply includes a short 5′ DNA extension that can be bound by the HUH-tag.

HUH endonucleases are enzymes that cleave and covalently attach to a single-stranded DNA (ssDNA) substrate in a sequence specific manner. These endonucleases are characterized by an active site including a pair of histidine (H) residues separated by a bulky hydrophobic residue (U). An HUH motif may alternately include one histidine, a bulky hydrophobic residue, and a glutamine used for cation coordination in place of the second histidine. Cation coordination triads are completed by a third residue, often glutamic acid or another histidine. This trio or pair of amino acids can coordinate a divalent cation, such as magnesium or manganese. When an HUH endonuclease binds a ssDNA substrate, the cation polarizes the ssDNA phosphate backbone, allowing the catalytic tyrosine to attack, cleave, and form a covalent linkage to the newly exposed 5′ end. HUH-tags suitable for use molecular tension systems of the present disclosure may be identified by the HUH motif described herein.

The use of HUH-tags in molecular tension sensors enables modular attachment of a broad range of ligands, enabling assessment of many different receptor-ligand interactions. In one or more embodiments, the ligand of interest may be a fusion protein with the HUH-tag. In other embodiments, the ligand of interest may be chemically ligated to the HUH-tag. Any ligand of interest may be suitable for use with a molecular tension sensor. Examples of potential ligands of interest include peptide hormones, growth factors, peptide toxins, ion channel inhibitors, integrin-binding linear or cyclic RGD peptides, cell-adhesion molecules, antibodies, scFvs, cadherins-binding molecules, selectin-binding molecules, or immunoglobulin-binding molecules.

Many different HUH-tags may be suitable for use with molecular tension sensors. Currently characterized HUH-tags are typically named for the source from which they are derived, and include wheat dwarf virus (WDV), porcine circovirus (PCV), duck circovirus (DCV), RepB, and tomato circovirus (TCV). Additional HUH-tags that may be compatible with use in molecular tension sensors are described in U.S. Pat. No. 10,717,773 and Tompkins, Kassidy J et al. “Molecular underpinnings of ssDNA specificity by Rep HUH-endonucleases and implications for HUH-tag multiplexing and engineering.” Nucleic acids research vol. 49, 2 (2021).

Strand rupture may be read out by measuring the presence of the ligand strand within the cell, for example, by fluorescence. In one or more embodiments, the anchor and/or ligand strand of a molecular tension sensor may include modifications to enable readout molecular tension. The ligand strand may include a fluorophore on the 3′ end, the 5′ end, and/or an internal nucleotide. The anchor strand may include a fluorophore on the 3′ end, the 5′ end, and/or an internal nucleotide. In some of these embodiments, the fluorophore may be a fluorescent protein. The fluorescent protein may be GFP, CFP, RFP, YFP, m Venus, tdTomato, mKate2, mCherry, or any other suitable fluorescent protein. In some of these embodiments, the fluorophore may be a small-molecule fluorophore. The small-molecule fluorophore may be CY5, CY5.5, CY3, FAM, fluorescein, HEX, TAMRA, ROX, an ATTO dye, an ALEXAFLUOR dye, or any suitable small-molecule fluorophore.

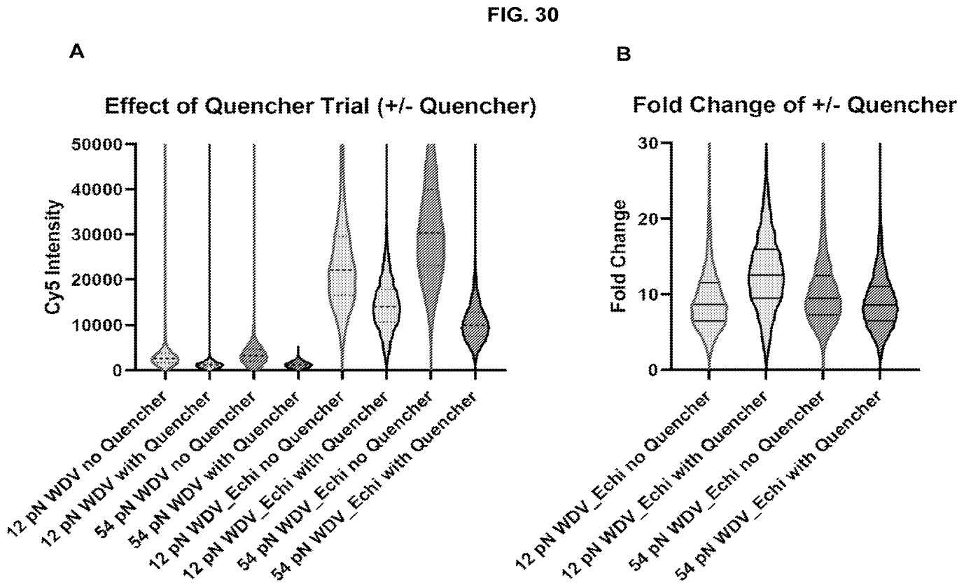

In one or more embodiments, a ligand strand may include a quencher. A quencher may help control for spurious detachment of the full molecular tension sensor duplex from the surface, as it shown in FIG. 30. The ligand strand may include a fluorescence quencher on the 3′ end, the 5′ end, and/or an internal nucleotide. The anchor strand may include a fluorescence quencher on the 3′ end, the 5′ end, and/or an internal nucleotide. The fluorescence quencher may be IOWA BLACK-FQ, IOWA BLACK-RQ, BLACK HOLE QUENCHER-1, BLACK HOLE QUENCHER-2, dabcyl, or any suitable fluorescence quencher.

In one or more embodiments, the first fluorophore and second fluorophore form a Förster resonance energy transfer pair. In some embodiments, the anchor strand or the ligand strand includes a bioluminescent protein, while the other strand includes a fluorophore. In some of these embodiments, the fluorophore and bioluminescent protein form a bioluminescence resonance energy transfer pair.

Strand rupture may alternately be read out by a downstream effect of the ligand strand being internalized. The ligand strand may not effect a cellular change in response to internalization, or it may cause a downstream cellular effect upon internalization. The change caused by ligand internalization is generally dictated by the molecules attached to the ligand strand. In one or more embodiments, the ligand strand may include molecules, such as proteins, in addition to the ligand. In one or more embodiments, the ligand strand includes any suitable molecule to enable a readout of strand rupture. In some embodiments, the ligand strand includes to one or more of luciferase, nanobodies, scFvs, other affinity molecules, peptides, protein tags, hormones, lipids, genome engineering reagents, transcription factors, fluorophores, small molecule drugs, cell cycle effectors, gene expression cassettes, siRNA, shRNA, mRNA, or other molecules.

In one or more embodiments, the anchor strand and/or the ligand strand may include additional nucleotide modifications. Nucleotide modifications may include, but are not limited to, amino modifiers, 3′ and/or 5′ phosphorylation, chemical spacer such as PEG moieties, phosphothioate bonds, conjugation modifications such as azide moieties, or a combination thereof. In one or more embodiments, the anchor strand and/or the ligand strand may include DNA nucleotides, RNA nucleotides, xenonucleic acid nucleotides such as peptide nucleic acid nucleotides or locked nucleic acid nucleotides, or a combination thereof.

In one or more embodiments, the anchor strand, the ligand strand, or both the anchor strand and the ligand strand includes peptide nucleic acid nucleotides. Peptide nucleic acids (PNAs) are synthesized polymers having nucleobases, such as adenine, thymine, guanine, uracil, and cytosine. Rather than a phosphate and ribose or deoxyribose backbone, peptide nucleic acids include an N-(2-aminoethyl)-glycine backbone. This backbone is not known to naturally occur, but is often desirable in synthetic contexts because of its resistance to nuclease activity. PNA can form a hybrid with DNA or RNA. Mismatches from canonical base pairs are typically better tolerated by PNA/DNA hybrids. PNAs are typically thought to be more stable and resistant to enzymatic and chemical degradation than DNA or RNA.

Interestingly, the inventors have found that the HUH tags described herein are able to react with PNA sequences as long as the required ori nucleobase sequence is present. The molecular tension sensors described herein are often used in environments including nucleases. In addition, it can be useful to know the exact length of a nucleic acid sequence used in a molecular tension sensor, as the length and composition of a hybridized complementary sequence typically informs the molecular strength of the sensor. Thus, in one or more embodiments, a molecular tension sensor includes one or more peptide nucleic acid nucleotides. In one or more of these such embodiments, the first nucleic acid strand includes entirely peptide nucleic acid nucleotides. In one or more embodiments, the second nucleic acid strand includes entirely peptide nucleic acid nucleotides. For example, the second nucleic acid strand may be a peptide nucleic acid and not include DNA or RNA nucleotides. It may be desirable for the second nucleic acid to include only PNA nucleotides because it is typically exposed to nucleases more than the first nucleic acid strand.

In one or more embodiments, the anchor strand and/or the ligand strand may include a nucleotide barcode. The nucleotide barcode may be 3-100 nucleotides in length, 10-50 nucleotides in length, 20-40 nucleotides in length, 3-20 nucleotides in length, 5-15 nucleotides in length, or 10 nucleotides in length. In one or more embodiments where multiple molecular tension sensors are used, each molecular tension sensor design may include a different nucleotide barcode sequence. In one or more embodiments, the nucleotide barcode may be flanked by amplification sites, wherein the amplification sites are compatible with polymerase chain reaction (PCR) amplification. In one or more embodiments, the nucleotide barcode may be flanked by restriction enzyme sites. In one or more embodiments, the anchor and/or ligand strand of a molecular tension sensor may include more than one barcode.

In some embodiments, the anchor strand may include a second HUH-tag. The second HUH-tag may be fused to another protein to facilitate specific binding and immobilization to a surface. Conjugating the anchor strand to a binding protein could allow immobilization of molecular tension sensor to specifically decorated surfaces. For example, conjugating the anchor strand of a molecular tension sensor to the collagen-binding protein may enable immobilization of the molecular tension sensor in a collagen gel.

In one or more embodiments, a molecular tension sensor may be supplied in the form of a pre-coated plate. The plate may be glass. The plate may be plastic, such as polystyrene. The plate may be coated in any suitable extracellular matrix component, such as fibronectin or collagen. The plate may be coated with fibronectin. The plate may be silanized.

In one or more embodiments, a molecular tension sensor may be supplied in the form of a kit. The kit may include one or more recombinantly produced molecular tension sensors conjugated to one or more ligands of interest, buffers, instructions for use.

In another aspect, this disclosure describes a method of preparing a molecular tension sensor. In one or more embodiments, a method of preparing a molecular tension sensor includes providing an anchor strand immobilized to a substrate, providing a ligand strand, wherein the ligand strand includes a region of nucleotides complementary to the anchor stand and a region of nucleotides recognizable by an HUH-tag, hybridizing the ligand strand and the anchor strand, wherein hybridizing includes annealing the region of complementary nucleotides to form a hybridized assembly, and reacting the ligand strand with an HUH-tag, wherein the HUH-tag includes a ligand of interest.

In some embodiments, the HUH-tag is reacted with the ligand strand after the ligand strand has been hybridized to the anchor strand. In some embodiments, the HUH-tag is reacted with the ligand strand before the ligand strand is hybridized to the anchor strand.

In some of these embodiments, the substrate is compatible with cell culture. The substrate may be a 96-well plate, a 24-well plate, a 12-well plate, a 6-well plate, a culture dish, or a culture flask. The substrate may be glass or it may be plastic. The substrate may be coated with a protein prior to immobilization of the anchor. For example, the substrate may be coated with neutravidin, streptavidin, albumin, fibronectin, or a combination thereof.

### Measuring and Quantifying Cellular Forces Using Molecular Tension Sensors

In one or more embodiments, molecular tension sensor may directly measure individual cells, rather than bulk properties of a plurality of cells. In one or more embodiments, RAD-TGTs enable readout of immobilized nucleic acid duplex rupture in individual cells of interest using flow cytometry and/or high-throughput sequencing.

A potential feature of the molecular tension sensors described herein, including the RAD-TGT system, is the ability to study molecular forces over time. In one or more embodiments, long-term studies of cellular tension may be carried out by adding HUH-ligands at variable timepoints after plating cells on RAD-TGTs that had not yet been reacted with an HUH-ligand. There are many HUH-tags compatible with RAD-TGTs and a large number of ligands of interest. In one or more embodiments, the RAD-TGT system may be multiplexed with at least three different ligands of interest, at least five different ligands of interest, at least ten different ligands of interest, or at least 50 different ligands of interest.

In another aspect, the present disclosure relates to methods of using a molecular tension sensor. A method of using a molecular tension sensor may be a method of measuring molecular forces in a cell. In one or more embodiments, a method includes providing a surface including the molecular tension sensor, contacting a cell with the surface, measuring the molecular forces in the cell, and interpreting the measurement.

In one or more embodiments, contacting includes culturing the cell on the surface. In one or more embodiments, measuring includes measuring the presence of the molecular tension sensor. In one or more embodiments, measuring includes measuring fluorescence. Measuring may include any method described herein, including fluorescent flow cytometry and nucleic acid sequencing.

As described above, the mechanical profile of a cell can provide information about the health and status of the cells. In one or more embodiments, a molecular tension sensor described herein can be used to investigate a cell. In one or more embodiments, a cell may be a cancer cell. For many cancers, it may be useful to use a molecular tension sensor to profile the mechanosome.

In one or more embodiments, a method or molecular tension sensor disclosed herein is used to investigate the mechanosome of a cell. In one or more of these such embodiments, the cell is a cancer cell. A cancer cell may be derived from any type of cancer including brain cancer (e.g., glioma, glioblastoma, astrocytoma), breast cancer, lung cancer, skin cancer, liver cancer, bone cancer, a cancer of the immune system, a blood cancer, ovarian cancer, testicular cancer, or kidney cancer. In one or more particular embodiments, the cancer cell is a glioblastoma cell.

A cancer cell may be an immortalized cancer cell, such as a stable cell line. Examples of stable cell lines include U251 cells, HEK293 cells, and HeLa cells. In one or more other embodiments, a cancer cell may be a primary cell. For example, a cancer cell may be derived from a patient sample such as a biopsy or a tumor. A cell may be human, primate, murine, ovine, hircine, or bovine.

### Flow Cytometry

In one or more embodiments, flow cytometry may be used as a sensitive method to readout ruptured and internalized fluorescent oligos of cells plated on molecular tension sensors. Flow cytometry may be advantageous in that it allows analysis of thousands of cells in minutes for as many as 30 fluorophores. Fluorescent flow cytometry of cells treated with molecular tension sensors may provide a measure of cellular forces proportional to fluorescence intensity. These measurements may be taken rather than, or in addition to, fluorescence microscopy imaging.

In one or more embodiments, fluorescence results from ligand-dependent delivery of the ruptured ligand strand into the cell of interest. In these embodiments, fluorescence of a fluorophore on the ligand strand may be measured directly. In one or more embodiments, measurement of the internalized fluorescence of molecular tension sensor components may be collected to measure of changes to cytoskeletal modulators.

In one or more embodiments, the readout may be sensitive enough to detect oligos conjugated to a single fluorophore in multiple cell lines without requiring additional amplification steps used in recent studies. In one or more embodiments, the sensitivity of a flow cytometry-based readout could be improved by attaching tandem fluorophores or even quantum dots.

In the body, cells exist in complex, three-dimensional environments and are in contact with other cells and extracellular matrix components on all sides. However, many cell culture systems use two-dimensional culture environments, such as plates or flasks. In these environments, cells typically grow in a monolayer and primarily receive mechanical signals from the base of the plate. Three-dimensional culture systems may be a more accurate recreation of mechanical extracellular matrix conditions than two-dimensional plated culture systems. However, traditional methods of measuring molecular tension often rely on microscopy techniques that are not compatible with three-dimensional culture systems. In one or more embodiments, the molecular tension sensors may be used in combination with three-dimensional culture systems. In one or more embodiments, molecular tension sensors may be incorporated into the matrix of a three-dimensional culture system. Cells cultured in a molecular tension sensor-populated three-dimensional culture system may be dissociated and analyzed using flow cytometry, sequencing, or both. In one or more embodiments, a three-dimensional cell culture system may be prepared with any suitable number of multiplexed molecular tension sensors.

### High-Throughput Sequencing

In one or more embodiments, molecular tension sensors allow high-throughput sequencing-based measurements of duplex rupture via ligand-dependent delivery of the ruptured ligand strand into the cell of interest. To measure internalization of the ligand strand, it is typically necessary to include a barcode with a known sequence that maybe amplified prior to sequencing. Following internalization, the ruptured DNA oligo may be detected by lysing cells, extracting the barcodes, amplifying the barcodes, and sequencing the captured barcodes. The ability to sequence captured barcodes in combination with NGS could permit further multiplexing of ligands and molecular tension sensors of variable tension tolerances. In one or more embodiments, a large number of molecular tension sensors may be conjugated to different ligands, wherein each ligand-conjugated molecular tension sensor includes a different DNA barcode. This type of approach may be useful in the context of large drug or CRISPR screens. This type of approach may also be used to screen large libraries of ligands recognizing patient T-cells or for whole genome CRISPR-knockout screens to identify key mechanosensors underlying a given mechanical phenotype.

In one or more embodiments, multiple types of analyses may be combined. For example, a molecular tension sensor may be designed with a ligand strand that includes both a barcode and a fluorescent label. Cells treated with this type of molecular tension sensor may be first analyzed using cell sorting, then they may be lysed, and the barcodes may be extracted, amplified, and sequenced to give a dual readout of flow cytometry and sequencing data. It should be noted that the analysis methods described herein may be performed in addition to any other suitable analysis of cellular tension, including but not limited to high-resolution fluorescence microscopy, traction force microscopy, and atomic force microscopy.

### EXEMPLARY EMBODIMENTS

Embodiment 1 is a molecular tension sensor including: a first nucleic acid strand nucleic acid; a second nucleic acid strand nucleic acid, wherein the second nucleic acid strand includes a region of at least five nucleotides that are complementary to a region of at least five nucleotides of the first nucleic acid strand; and an HUH-tag.

Embodiment 2 is the molecular tension sensor of Embodiment 1, further including a ligand.

Embodiment 3 is the molecular tension sensor of Embodiment 2, wherein the ligand and the HUH-tag include a fusion protein.

Embodiment 4 is the molecular tension sensor of Embodiment 2, wherein the ligand and the HUH-tag are chemically ligated.

Embodiment 5 is the molecular tension sensor of any of Embodiments 2-4, wherein the ligand includes a nanobody, an antibody, or an scFv.

Embodiment 6 is the molecular tension sensor of any of Embodiments 1-5, wherein the first nucleic acid strand is immobilized to a surface.

Embodiment 7 is the molecular tension sensor of Embodiment 6, wherein the first nucleic acid strand is immobilized to the surface by an affinity interaction.

Embodiment 8 is the molecular tension sensor of Embodiment 6, wherein the first nucleic acid strand is immobilized to the surface by a biotin-avidin interaction.

Embodiment 9 is the molecular tension sensor of Embodiment 6, wherein the first nucleic acid strand is immobilized to the surface by adsorption.

Embodiment 10 is the molecular tension sensor of any of Embodiments 1-9, wherein the first nucleic acid strand is 5 nucleotides to 80 nucleotides in length.

Embodiment 11 is the molecular tension sensor of any of Embodiments 1-10, wherein the second nucleic acid strand is 20 nucleotides to 100 nucleotides in length.

Embodiment 12 is the molecular tension sensor of any of Embodiments 1 to 11, wherein the second nucleic acid strand includes a fluorophore.

Embodiment 13 is the molecular tension sensor of Embodiment 12, wherein the fluorophore is a small-molecule fluorophore.

Embodiment 14 is the molecular tension sensor of Embodiment 12 or Embodiment 13, wherein the fluorophore is CY5, CY5.5, or CY3.

Embodiment 15 is the molecular tension sensor of any of Embodiments 1 to 14, wherein first nucleic acid strand includes a fluorescence quencher.

Embodiment 16 is the molecular tension sensor of any of Embodiments 1 to 15, wherein the region of complementary nucleotides of the second nucleic acid strand is 10 nucleotides to 30 nucleotides in length.

Embodiment 17 is the molecular tension sensor of any of Embodiments 1 to 16, wherein the region of complementary nucleotides of the second nucleic acid strand is at least 75%, at least 80%, at least 90%, or at least 95% complementary to a region of the first nucleic acid strand.

Embodiment 18 is the molecular tension sensor of any of Embodiments 1 to 7, wherein the region of complementary nucleotides of the second nucleic acid strand is 100% complementary to a region of the first nucleic acid strand.

Embodiment 19 is the molecular tension sensor of any of Embodiments 1 to 6, wherein the second nucleic acid strand includes a barcode sequence.

Embodiment 20 is the molecular tension sensor of Embodiment 19, wherein the barcode includes 5 nucleotides to 15 nucleotides.

Embodiment 21 is the molecular tension sensor of any of Embodiments 1 to 20, wherein the first nucleic acid strand, the second nucleic acid strand, or both includes modified nucleotides.

Embodiment 22 is a plate for analysis of molecular tension in cultured cells including the molecular tension sensor of any of Embodiments 1 to 21.

Embodiment 23 is the plate of Embodiment 22, wherein the plate is glass.

Embodiment 24 is the plate of Embodiment 22, wherein the plate is plastic, such as polystyrene.

Embodiment 25 is the plate of Embodiment 22, wherein the plate is wherein the plate is coated with fibronectin.

Embodiment 26 is the plate of Embodiment 22, wherein the plate is wherein the plate is silanized.

Embodiment 27 is the plate of Embodiment 22, wherein the plate is coated with an affinity molecule, such as neutravidin or streptavidin.

Embodiment 28 is a kit for analysis of molecular tension in cultured cells, the kit including the plate of any of Embodiments 22 to 27, one or more buffers, and instructions for use.

Embodiment 29 is a kit for analysis of molecular tension, the kit including the molecular tension sensor of any of Embodiments 1 to 20, one or more buffers, and instructions for use.

Embodiment 30 is a method of preparing a molecular tension sensor, the method including providing a first nucleic acid strand immobilized to a substrate; providing a second nucleic acid strand, wherein the second nucleic acid strand includes a region of nucleotides complementary to the anchor stand and a region of nucleotides recognizable by an HUH-tag; hybridizing the second nucleic acid strand and the first nucleic acid strand, wherein hybridizing includes annealing the region of complementary nucleotides to form a hybridized assembly; and reacting the second nucleic acid strand with an HUH-tag, wherein the HUH-tag includes a ligand.

Embodiment 31 is a method of measuring molecular forces in a cell, the method including: providing a surface including the molecular tension sensor of any of Embodiments 1 to 20; culturing a cell on the surface; measuring the molecular forces in the cell, wherein measuring includes measuring the presence of the molecular tension sensor; and interpreting the measurement of the presence of the molecular tension sensor as it relates to molecular forces.

Embodiment 32 is the method of Embodiment 31, wherein measuring the presence of the molecular tension sensor includes measuring fluorescence.

Embodiment 33 is the method of Embodiment 32, wherein fluorescence is measured using flow cytometry.

Embodiment 34 is the method of Embodiment 32, wherein fluorescence is measured using high-resolution fluorescence microscopy

Embodiment 35 is the method of Embodiment 31, further including lysing the cell, extracting DNA from the cell, amplifying the DNA, and sequencing the DNA.

Embodiment 36 is the method of Embodiment 35, wherein measuring the presence of the molecular tension sensor includes analyzing DNA sequencing results.

Embodiment 37 is the method of any of Embodiments 31 to 36, wherein the cell includes a cancer cell.

Embodiment 38 is the method of Embodiment 37, wherein the cancer cell includes a glioma cell.

Embodiment 39 is the method of Embodiment 37, wherein the cancer cell includes an immortalized cell.

Embodiment 40 is the method of Embodiment 37, wherein the cancer cell includes a primary cell.

Embodiment 41 is the molecular tension sensor of any of Embodiments 1 to 20, wherein the first nucleic acid strand, the second nucleic acid strand, or both includes one or more peptide nucleic acid nucleotides.

Embodiment 42 is the molecular tension sensor of Embodiment 41, wherein the second nucleic acid strand includes a peptide nucleic acid and does not include DNA or RNA nucleotides.

As used herein, the terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

As used herein, the term “or” is generally employed in its usual sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

Herein, “up to”, “at most”, “not more than”, or “not greater than” a number (for example, up to 50) includes the number (for example, 50). Herein, “at least”, or “not less than” a number (for example, at least 50) includes the number (for example, 50).

The term “in the range” or “within a range” and similar statements includes the endpoints of the stated range.

For any method disclosed herein that includes discrete steps, the steps may be conducted in any feasible order. And, as appropriate, any combination of two or more steps may be conducted simultaneously.

Any reference to standard methods (e.g., recombinant protein production, column chromatography) refer to the most recent available version of the method at the time of filing of this disclosure unless otherwise indicated.

The term “nucleic acid” as used herein refers to a polymer containing at least two nucleotides (i.e., deoxyribonucleotides or ribonucleotides) in either single- or double-stranded form and includes DNA and RNA. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. Nucleic acids include nucleic acids containing known nucleotide analogs or modified backbone residues or linkages or modified sugar residues, or non-canonical/chemically-modified nucleobases and combinations thereof, which are synthetic, naturally occurring, and non-naturally occurring, and which have similar binding properties as the reference nucleic acid. Examples of such analogs and/or modified residues include, without limitation, phosphorothioates, phosphoramidates, methyl phosphonates, chiral-methyl phosphonates, 2′-O-methyl ribonucleotides, and peptide-nucleic acids (PNAs). For purposes of percent sequence identity between an RNA sequence and a DNA sequence, uracil bases in the RNA are to be considered identical to thymine bases in DNA sequences.

The term “complementary” is used throughout this application to describe two related nucleic acid sequences that may form a double-stranded complex of a first 5′ to 3′ “top” strand and a second 3′ to 5′ “bottom” strand.

As used herein, the terms “protein,” “polypeptide,” and “peptide” are used interchangeably and refer to a polymer of amino acid residues linked via peptide bonds and which may be composed of two or more polypeptide chains. The terms “polypeptide,” “protein,” and “peptide” refer to a polymer of at least two amino acid monomers joined together through amide bonds. An amino acid may be the L-optical isomer or the D-optical isomer. More specifically, the terms “polypeptide,” “protein,” and “peptide” refer to a molecule composed of two or more amino acids in a specific order; for example, the order as determined by the base sequence of nucleotides in the gene or RNA coding for the protein. Proteins are essential for the structure, function, and regulation of the body's cells, tissues, and organs, and each protein has unique functions. Examples of proteins include hormones, enzymes, antibodies, and any fragments thereof. In some cases, a protein can be a portion of the protein, for example, a domain, a subdomain, or a motif of the protein. In some cases, a protein can be a variant (or mutant) of the protein, wherein one or more amino acid residues are inserted into, deleted from, and/or substituted into the naturally occurring (or at least a known) amino acid sequence of the protein. A protein or a variant thereof can be naturally occurring or recombinant. Methods for detection and/or measurement of polypeptides in biological material are well known in the art and include, but are not limited to, Western blotting, flow cytometry, enzyme-linked immunosorbent assay (ELISA), radioimmunoassay (RIA), and various proteomics techniques, such as mass spectrometry. An exemplary method to measure or detect a polypeptide is an immunoassay, such as an ELISA. This type of protein quantitation can be based on an antibody capable of capturing a specific antigen, and a second antibody capable of detecting the captured antigen.

Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

The above summary of the present invention is not intended to describe each disclosed embodiment or every implementation of the present invention. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list.

### EXAMPLES

The present invention is illustrated by the following examples. It is to be understood that the particular examples, materials, amounts, and procedures are to be interpreted broadly in accordance with the scope and spirit of the invention as set forth herein. Throughout these examples, “room temperature” refers to a temperature of 18° C. to 26° C., preferably 22° C. to 24° C.

TABLE 1
Reagents used in the examples

| Reagent | Source |
|---|---|
| 96-well plates | |
| Biotin-BSA | |
| Ni-NTA affinity resin | THERMO FISHER, for example |
| | 88221, for example |
| Size exclusion chromatography column | BIORAD, for example |
| | Enrich SEC70, for example |
| E. coli for protein expression | THERMO FISHER, for example |
| BL21 (DE3) E. coli, for example | EC0114, for example |
| Annealing buffer | |
| 10 mM Tris pH 7.5 | SIGMA ALDRICH, |
| | for example T2319 |
| 50 mM NaCl | SIGMA ALDRICH, |
| 1 mM EDTA | for example S9888 |
| | SIGMA ALDRICH, |
| | for example E8008 |
| Reaction buffer | |
| 50 mM HEPES pH 8.0 | SIGMA ALDRICH, |
| | for example 83264 |
| 50 mM NaCl | SIGMA ALDRICH, |
| | for example S9888 |
| 1 mM MnCl₂ | SIGMA ALDRICH, |
| | for example 13220 |
| U251 cells | SIGMA ALDRICH, for example |
| | 09063001, for example |
| CHO-K1 cells | AMERICAN TYPE CULTURE |
| | COLLECTION (ATCC) |
| | CCL-61 |
| FuGENE Transfection Reagent | PROMEGA |
| Dulbecco's modified eagle medium | CORNING |
| | 10027CV |
| Fetal bovine serum | R&D SYSTEMS |
| | S11150 |
| Penicillin/streptomycin | GIBCO |
| | 15070063 |
| F-12K medium | ATCC |
| | 302004 |
| Trypsin | Gibco |
| | 25200056 |
| Puromycin | |
| Opti-MEM Reduced Serum Media | GIBCO |
| | 31985062 |
| Automated cell counter | INVITROGEN |
| | Countess II |
| Para-amino-blebbistatin | CAYMAN CHEMICAL |
| | 22699 |
| Dimethyl sulfoxide (DMSO) | INVITROGEN |
| | D12345 |
| Phosphate buffered saline (PBS) | THERMO FISHER, for example |
| | 10010023 |
| Flow Cytometer | ACCURI C6 PLUS |
| | BD |
| 1x passive lysis buffer | PROMEGA |
| | E1941 |
| Fluorobrite DMEM | GIBCO |
| | A1896701 |
| Fluorescent microscope | EVOS FL AUTO |

TABLE 2
DNA sequences used in the examples

| Name | Sequence (5′ to 3′) |
|---|---|
| Fluorescently Labeled Ligand | GCTATAAACTCACCGTAATTTTTTGGCCCGCAGCGA |
| Strand | CCACCCTTT/3Cyy5Sp/- (SEQ ID NO: 1) |
| Quencher Ligand Strand | GCTATAAACTCACCGTAATTTTTTGGCCCGCAGCGA |
| | CCACCCTTT/3IAbRQSp/ (SEQ ID NO: 1) |
| Unlabeled Anchor Strand | GGGTGGTCGCTGCGGGCC (SEQ ID NO: 2) |
| 12 pN Quencher Anchor | /5IabRQ/GGGTGGTCGCTGCGGGCC/3Bio/- (SEQ ID |
| Strand | NO: 2) |
| 12 pN Unlabeled Anchor | GGGTGGTCGCTGCGGGCC/3Bio/ (SEQ ID NO: 2) |
| Strand | |
| 12 pN Fluorescently Labeled | /5CY5/GGGTGGTCGCTGCGGGCC/3Bio/ (SEQ ID NO: 2) |
| Anchor Strand | |
| 54 pN Quencher Anchor | /5IabRQ/iBiodT/GGGTGGTCGCTGCGGGCC (SEQ ID |
| Strand: | NO: 2) |
| Barcoded Ligand Strand 1 | GCTATAAACTCACCGTAATTTTTTGGCCCGCAGCGA |
| | CCACCCTTTTCTCTCTTGGCGTCATCGTGTACCGG |
| | (SEQ ID NO: 3) |
| Barcoded Ligand Strand 2 | GCTATAAACTCACCGTAATTTTTTGGCCCGCAGCGA |
| | CCACCCTTTAGACTCTTGGCGTCATCGTGTACCGG |
| | (SEQ ID NO: 4) |
| Barcoded Ligand Strand 3 | GCTATAAACTCACCGTAATTTTTTGGCCCGCAGCGA |
| | CCACCCTTTTCTGAGTTGGCGTCATCGTGTACCGG |
| | (SEQ ID NO: 5) |
| Forward Amplicon Primer | ACACTCTTTCCCTACACGACGCTCTTCCGATCTCTC |
| | ACCATGTGGTGACGCCAGAATTTGCCGCAATACAC |
| | AGTTTACGCCGTTCGGTCAGCTTGGTATCCGTAGCG |
| | CAGCGACCACCCTTT (SEQ ID NO: 6) |
| Reverse Amplicon Primer | GACTGGAGTTCAGACGTGTGCTCTTCCGATCTGAGA |
| | AACCTAAGCAGACTTCTCCTGGTCGATGATTGATAA |
| | GGGTCTCGGAATGTCCCCGGTCGCATGGTTCCGGTA |
| | CACGATGACGCC (SEQ ID NO: 7) |
| gRNA for TLN1 | AACUGUGAAGACGAUCAUGG (SEQ ID NO: 8) |
| gRNA for CD44 | GAATACACCTGCAAAGCGGC (SEQ ID NO: 9) |

### Example 1

This example describes that flow cytometry detects rupture and delivery of immobilized RAD-TGTs.

### Results

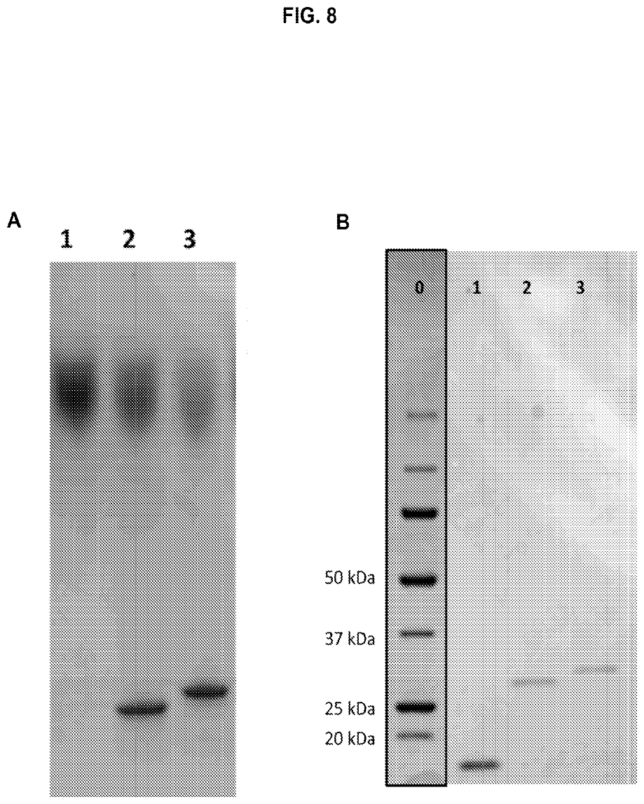

RAD-TGTs were first assembled as shown in FIG. 2. Echistatin was recombinantly expressed as a fusion protein with WDV (WDV-Echi) and purified. WDV not fused to another protein was also recombinantly expressed and purified. WDV or WDV-Echi were each reacted with the anchor strand-ligand strand duplex (sequences shown in Table 2). A native gel showing that WDV is able to bind to the anchor strand-ligand strand duplex is shown in FIG. 8. A 1 μM solution of each RAD-TGT was then immobilized on a glass plate coated with biotin-BSA, which had been previously incubated with neutravidin.

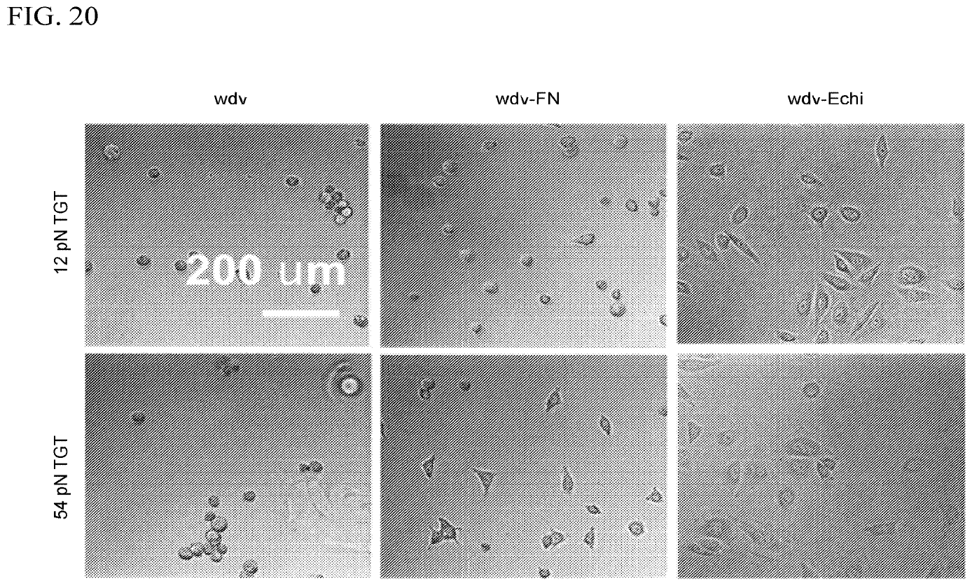

These experiments demonstrated that CHO-K1 cells failed to adhere to surfaces coated with 12 pN TGTs but could adhere to surfaces coated with 54 pN TGTs, suggesting that adhesion requires integrins to exert more than 12 pN of force. These results were recapitulated using the WDV-FN ligands conjugated to RAD-TGTs. These results were recapitulated using the WDV-FN ligands conjugated to RAD-TGTs (FIG. 20). Interestingly, CHO-K1 cells adhered to both 12 and 54 pN WDV-Echi surfaces. This is likely attributed to the higher affinity of echistatin for integrins and/or the wider array of integrin subtypes bound by echistatin than cyclic-RGD or FN, which may lead to adhesion in different force regimes as was demonstrated for α4β1 integrins in recent studies34. Nevertheless, these results indicate that the assembled TGTs are functional and that surface preparations are equivalent to prior studies.

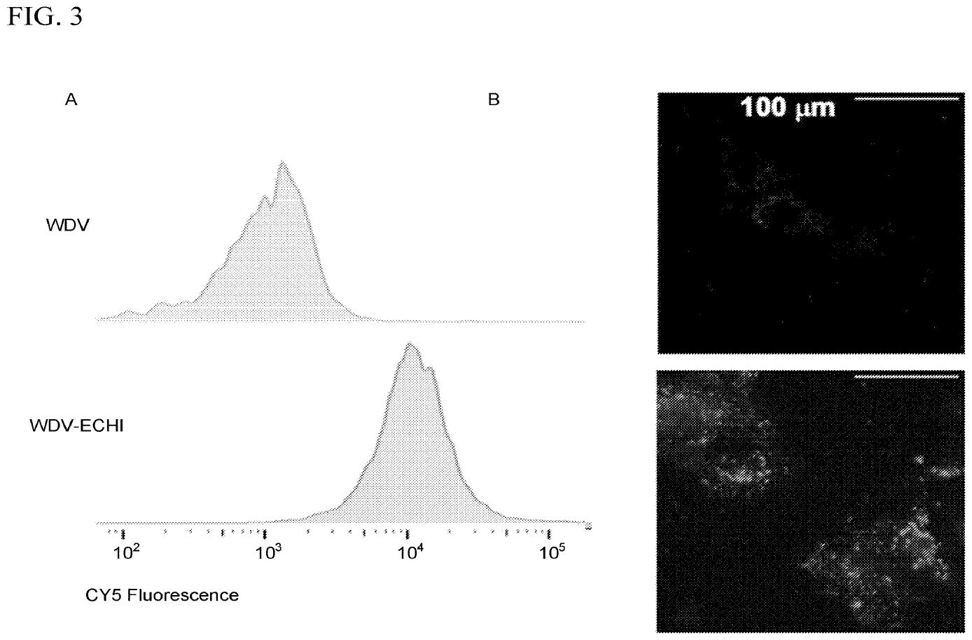

Next, cells were analyzed to determine whether flow cytometry could be used to detect TGT rupture. Approximately 15,000 cells were plated in each well in serum-free media and incubated for 1-4 hours. At each desired timepoint, the cells were trypsinized and analyzed by flow cytometry. RAD-TGTs were first tested on CHO-K1 cells. Flow cytometry of the CHO-K1 cells two hours after plating on RAD-TGTs shows a difference between the negative control WDV and WDV-Echi, with the fold change of median fluorescence being about 10-fold as shown in FIG. 3A. Two distinct and relatively narrow populations are observed. This suggests that a homogeneous population of cells plated on the RAD-TGTs were exerting tension on the surface. High-resolution fluorescence microscopy images shown in FIG. 3B were also collected to support the flow cytometry results.

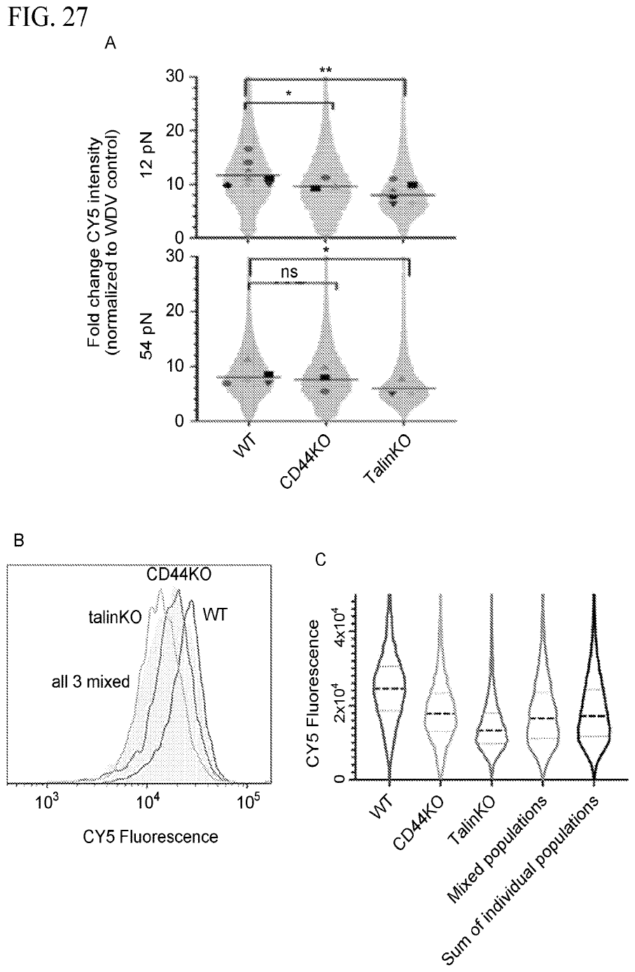

In addition, the RAD-TGT fluorescence histograms of the three cell lines resulted in distinct, separated peaks (FIG. 27B). A mixture of the three cell lines were plated and the fluorescence envelope resembles the sum of the three individual populations (FIG. 27B). Indeed, violin plots of the cumulative fluorescence plots of the three

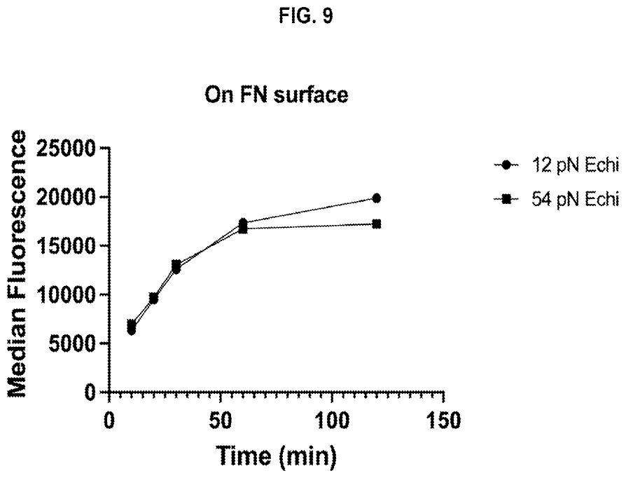

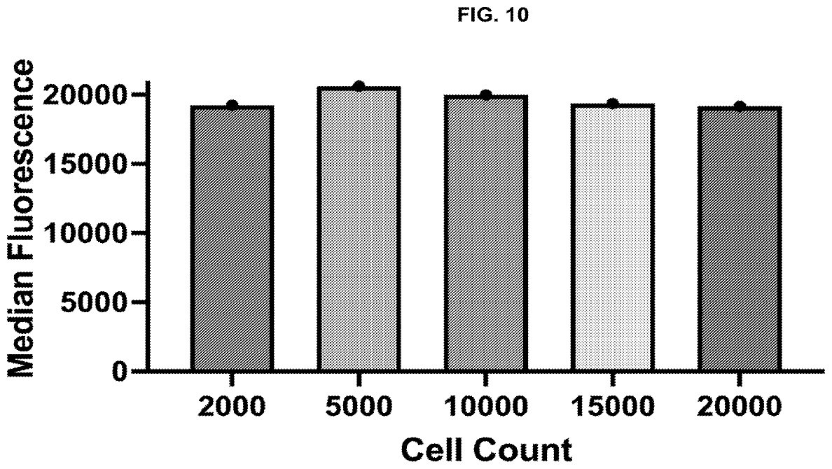

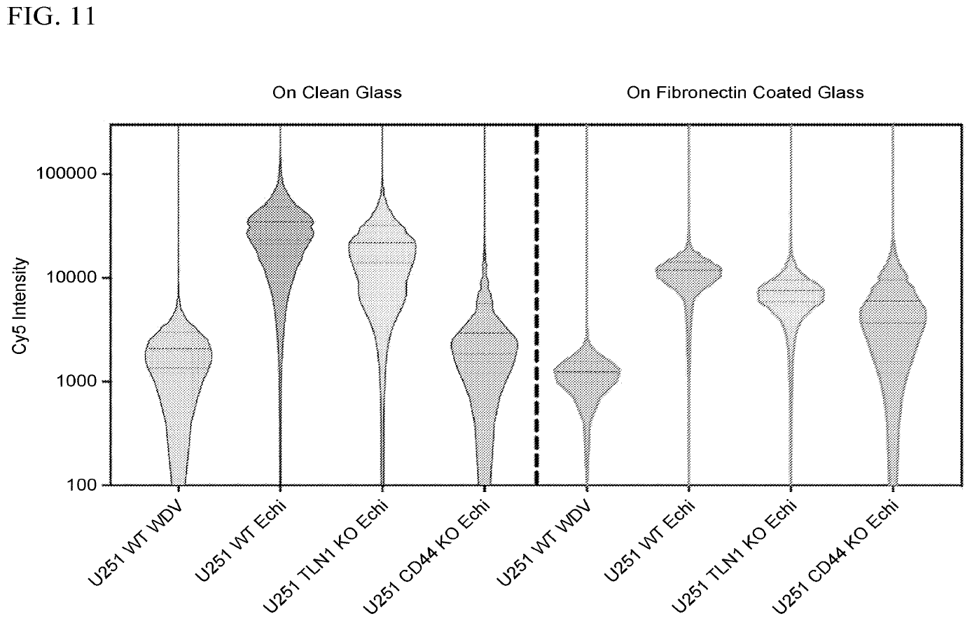

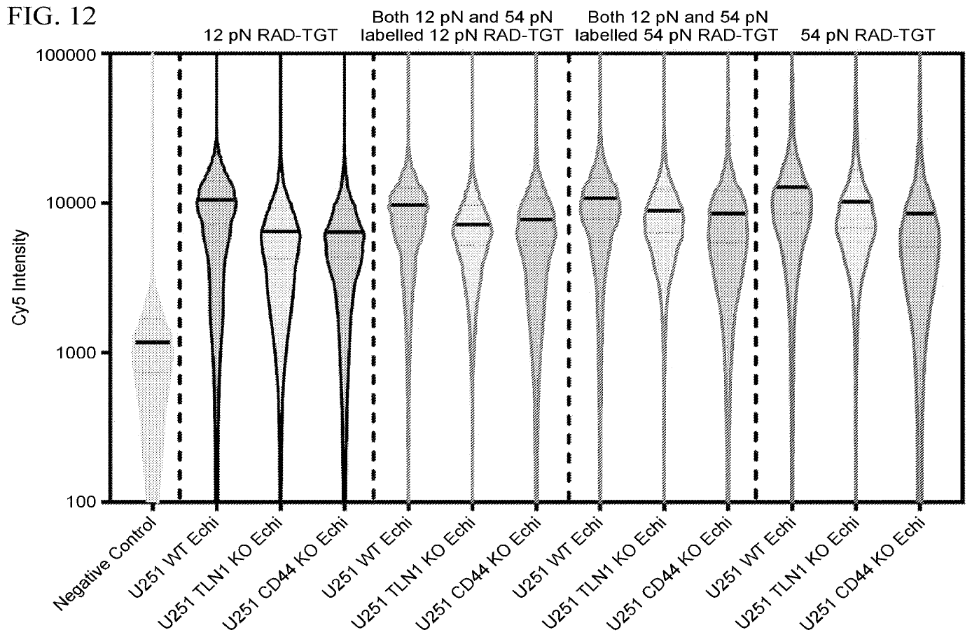

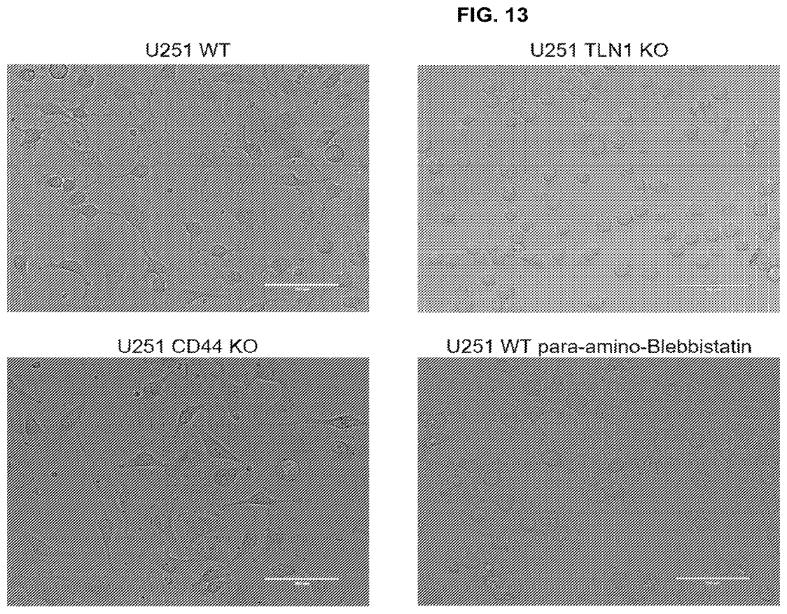

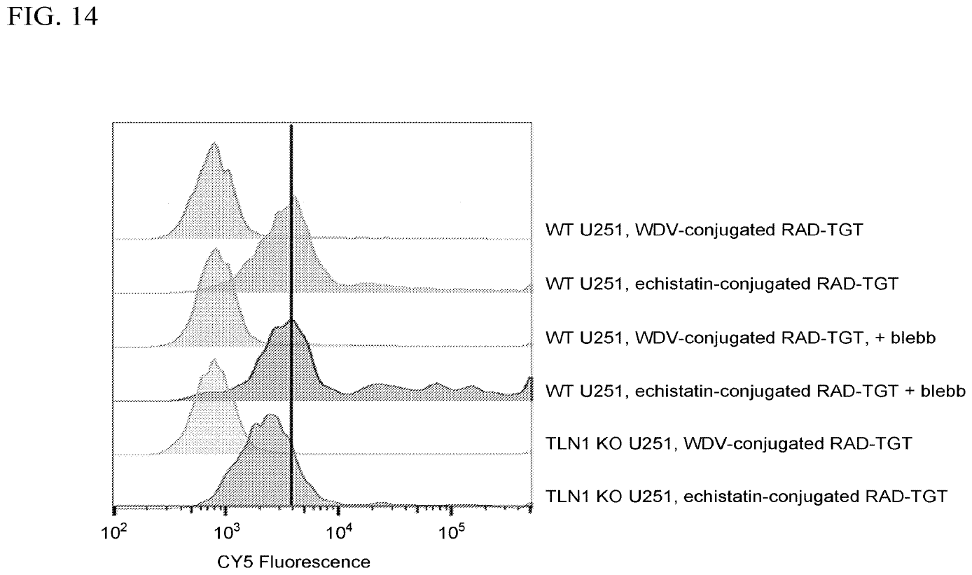

The ligand strand was labeled with CY5 and the anchor strand was labeled with a quencher to allow a gain-of-fluorescence readout upon RAD-TGT rupture. The CHO-K1 cells plated on WDV-Echi showed a strong fluorescence footprint under the cells, while WDV alone shows very little fluorescence. Additional characterization and optimization of the RAD-TGT protocol confirmed that internalized fluorescence increases with time after cell plating up to about 2 hours as shown in FIG. 9, and that overall median fluorescence does not depend strongly on cell density as shown in FIG. 10. This protocol was repeated fibronectin-coated surfaces and surfaces with mixed 12pN and 54 pN TGTs to clarify whether the observed results were not confounded by differences in cell behavior on 12pN TGTs. These results are shown in FIG. 11 and FIG. 12. Generally, all surface preps result in the same trends in the data and cells adhering well after 2 hours with varying morphologies as shown in FIG. 13. Initial attempts using commercial polystyrene neutravidin coated plates resulted in similar trends in median fluorescence as glass-bottom plates but an overall broader distribution of fluorescence as shown in FIG. 14, which was attributed to non-specific adsorption to polystyrene.

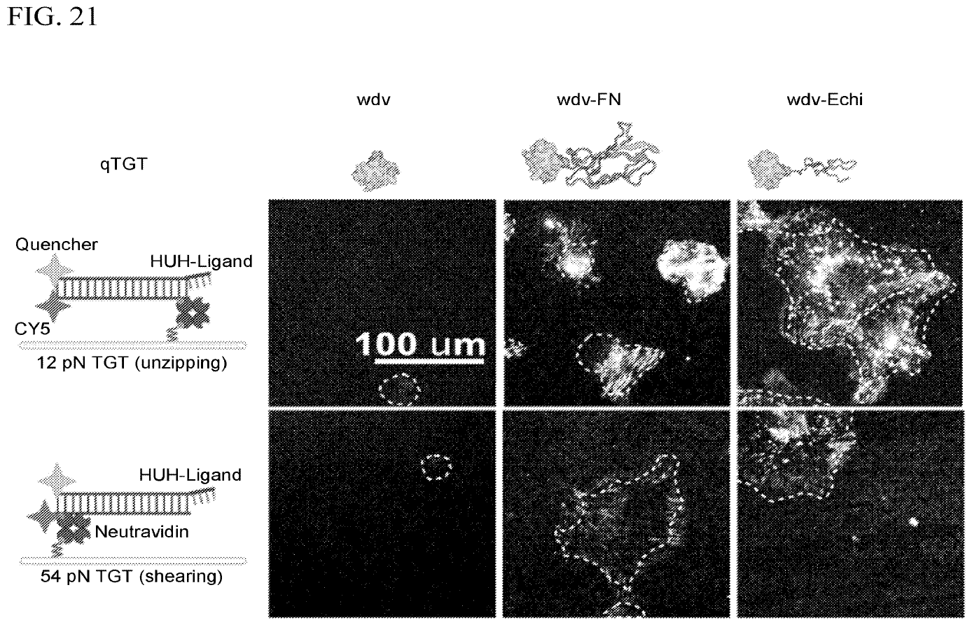

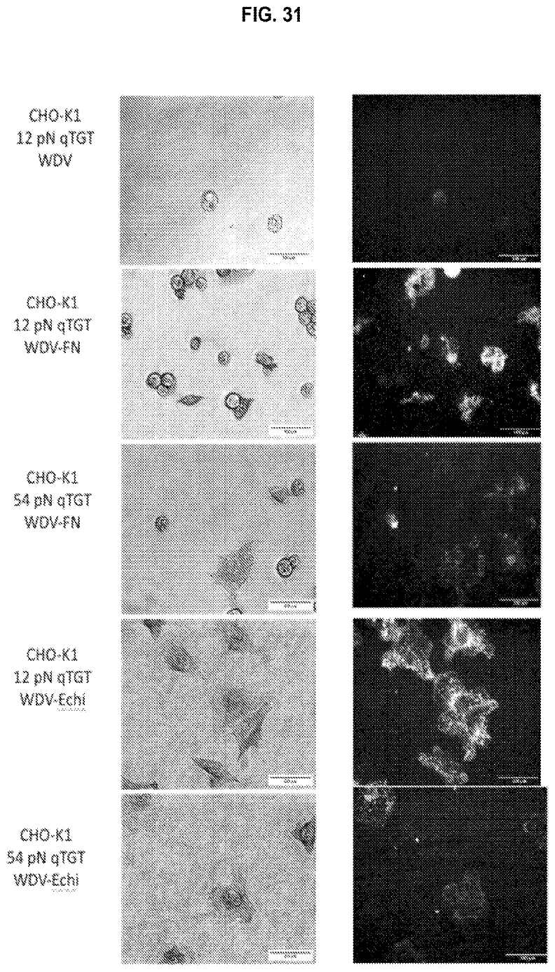

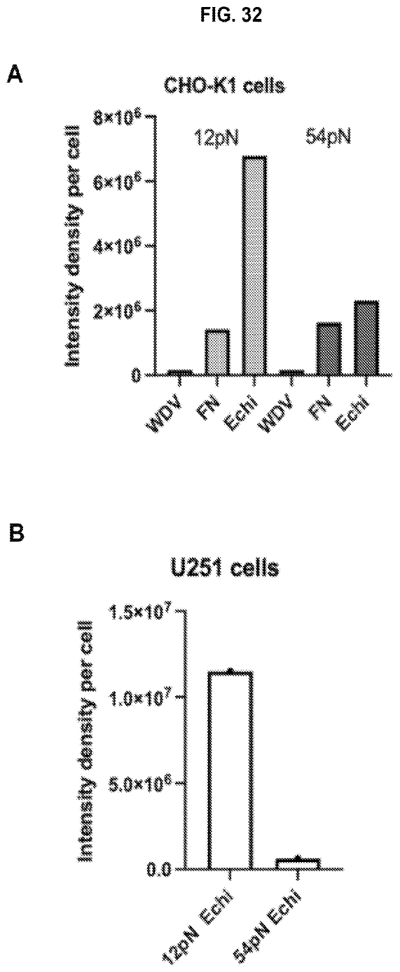

TGT rupture was measured via a traditional fluorescence microscopy readout using quenched TGTs (qTGTs). Briefly, qTGTs are similar to RAD-TGTs, but the fluorophore and quencher locations are inverted, thus allowing for a gain of fluorescence on the surface of the plate following rupture. CHO-K1 cells plated on 12 and 54 pN qTGTs conjugated to WDV adhered poorly and resulted in no fluorescence signal (FIG. 21, FIG. 31). Cells plated on the WDV-FN qTGTs resulted in rupture patterns similar to previous studies. Briefly, cells on the 12 pN surface struggled to adhere but caused robust rupture uniformly across the small cell footprint, while cells on 54 pN qTGTs adhered well but ruptured qTGTs in a streak pattern caused by motile focal adhesions (FIG. 21). This behavior corresponds to a higher rupture ratio of 12pN TGTs but an overall lower cumulative fluorescence intensity given the small cell footprint compared to 54pN TGTs, as corroborated by quantification of cumulative fluorescence intensity (FIG. 32). Intriguingly, cells plated on 12 and 54 pN TGTs conjugated to WDV-Echi, where cells adhere similarly, show both streaks and fluorescent puncta with a higher cumulative fluorescence signal across the cell footprint for 12pN than 54pN TGTs (FIG. 31, FIG. 32).

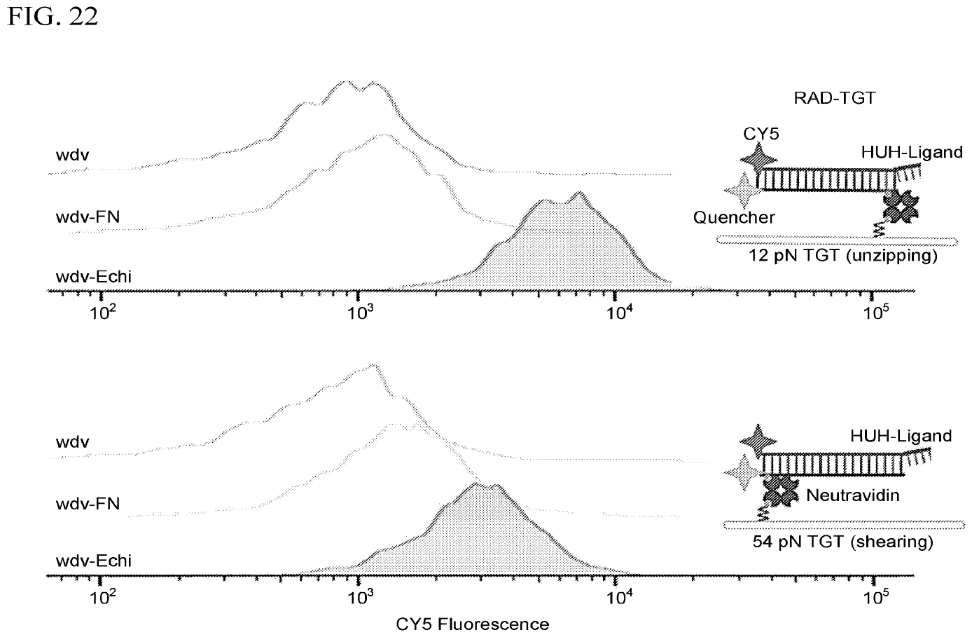

CHO-K1 cells were plated on both 12 and 54 pN RAD-TGT surfaces and then analyzed after 90-minute incubation (FIG. 22). Two measures are typically used to quantify TGT rupture using imaging; rupture ratio and integrated intensity for a given cell footprint. Rupture ratio readout is not thought to be influenced by cell size, and represents all rupture events, rather than the number of cells having a rupture event. Flow cytometry is considered to be a measure of the number of cells having a rupture event. Both metrics are considered valid and typically are closely aligned across conditions. Flow cytometry of the CHO-K1 cells showed a modest increases in median fluorescence for the WDV-FN ligands compared to WDV alone. The 12pN TGT showed lower fluorescence than 54 pN, likely due to the lack of cell adhesion under these conditions, as has been observed previously. Generally, literature studies show 12 pN TGTs are ruptured more readily than 54 pN TGTs fluorescence, but only under conditions where cell adhesion is facilitated by mixing 12 pN TGTs with TGTs of higher tension tolerance or by coating surfaces with fibronectin. Indeed, when WDV-Echi was conjugated to TGTs where cells adhere without assistance on both 12 and 54 pN TGTs, the fold change of CY5 median fluorescence intensity compared to WDV control was threefold and sevenfold for the 54 and 12 pN RAD-TGTs, respectively. The fluorescence histograms observed from rupture and delivery of RAD-TGTs are narrow, with distinct shifts in median fluorescence of the population compared to WDV alone negative controls. In contrast, other studies detecting cellular forces by flow cytometry of microparticles containing DNA tension probes interacting with cells of interest instead report a modest broadening of the population in response to cellular tension. This suggests that a homogeneous population of cells plated on the RAD-TGTs exerts tension on the surface.

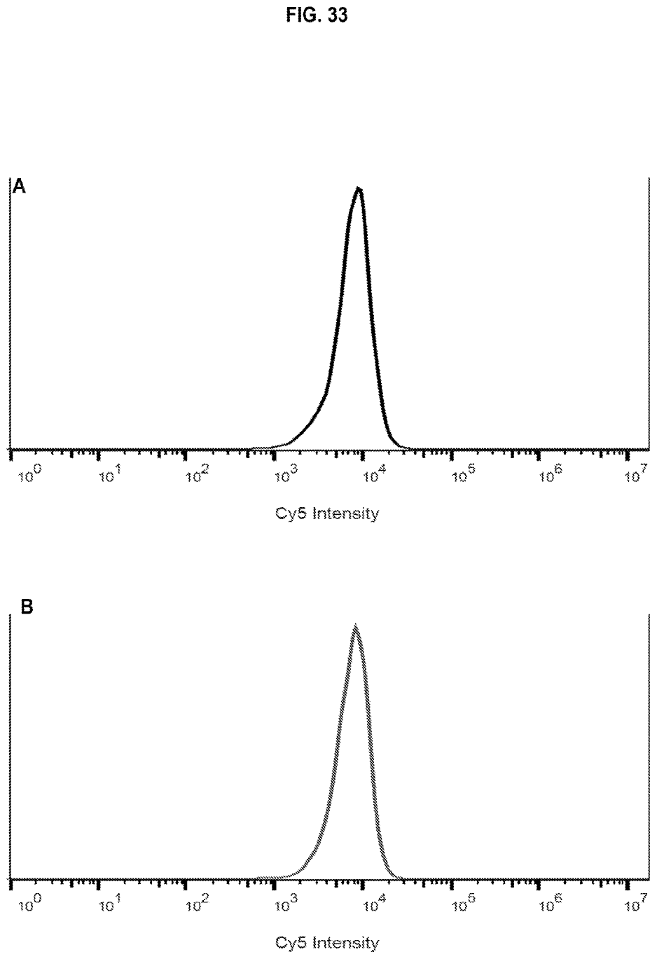

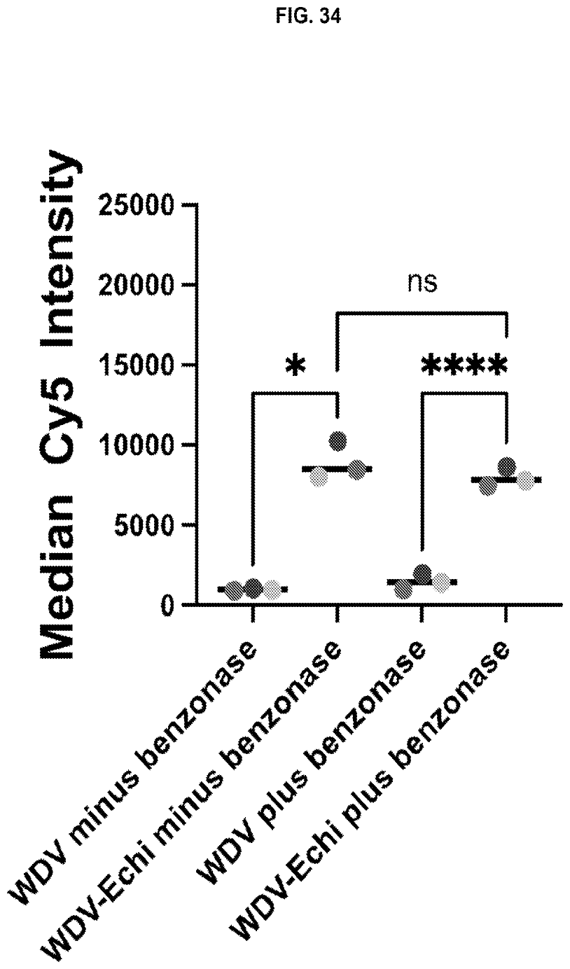

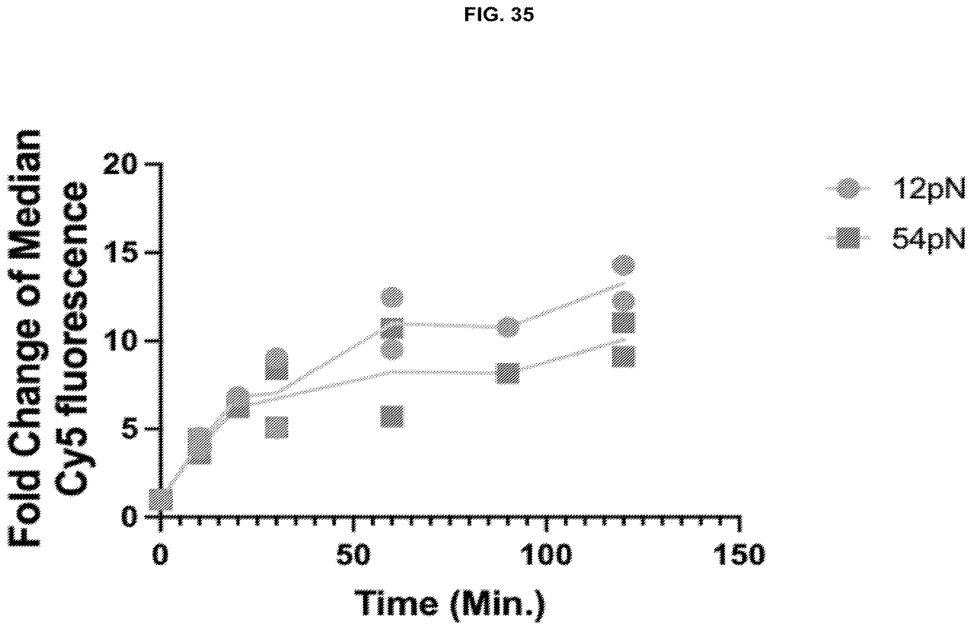

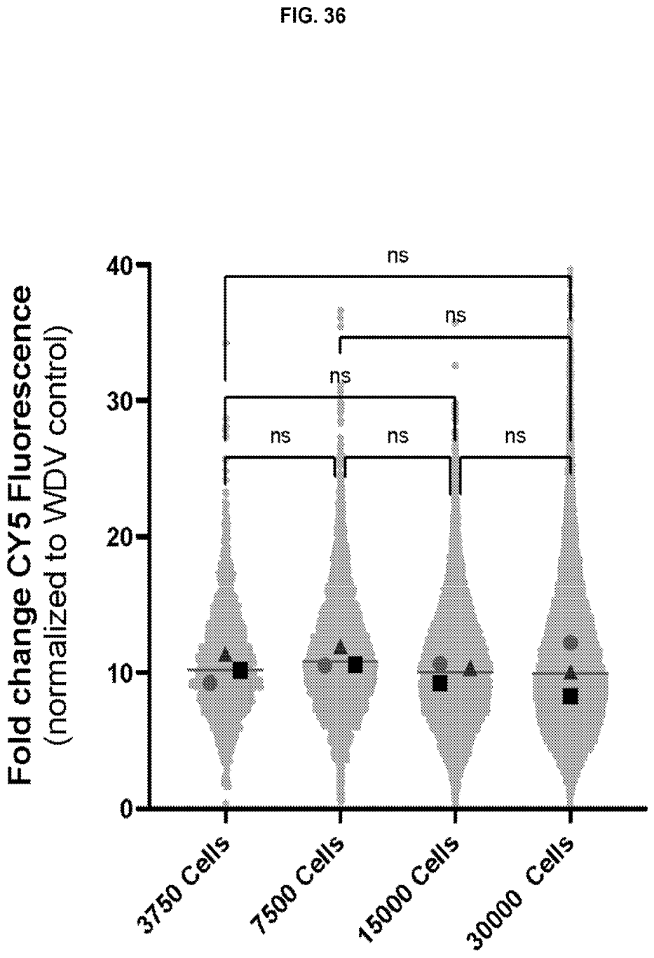

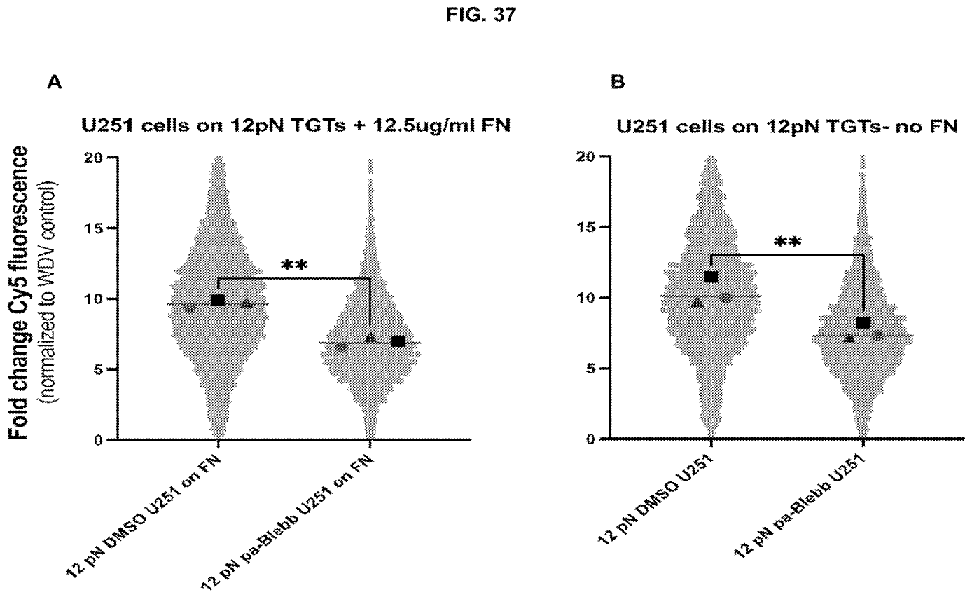

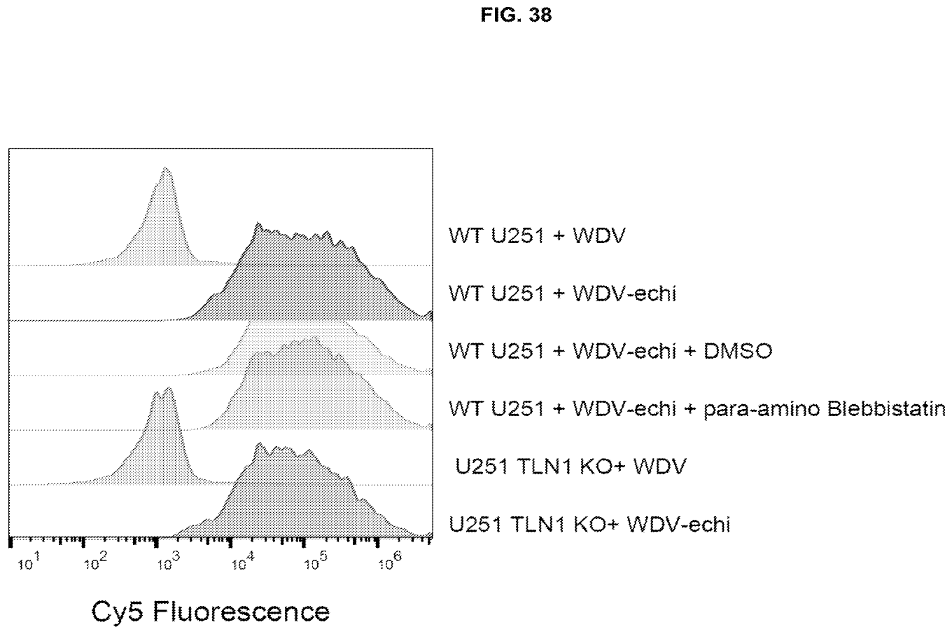

To determine whether the oligo was fully internalized or if some remained bound to the surface of the cell, a common assay was used to assess DNA origami delivery. Briefly, cells were plated on RAD-TGTs. Following incubation, cells were treated with a promiscuous nuclease or vehicle control and then analyzed via flow cytometry. There was a slight decrease in fluorescence following nuclease treatment (FIG. 33, FIG. 34), indicating that indeed a large percentage of the oligo delivered to the cell (~90%) was indeed internalized. Additional characterization and optimization of the RAD-TGT protocol confirmed that internalized fluorescence increases with time after cell plating up to about 2 hours (FIG. 35) and that overall median fluorescence does not depend strongly on cell density (FIG. 36). Other measurements were performed, such as including fibronectin in the coating protocol to ensure the results were not confounded by differences in cell behavior following TGT rupture (FIG. 37). Generally, all surface preps resulted in the same trends in the data and cells adhering well after two hours with varying morphologies. It should be noted that experiments were attempted on commercially available neutravidin-coated polystyrene plates, and the resulting fluorescence profiles showed non-specific adsorption and volatile and noisy readouts (FIG. 38). Thus, most other experiments were performed using 96-well glass-bottom plates.

### Experimental Methods

HUH-Ligand Preparation: HUH variants were expressed in E. coli and purified via subsequent Ni-NTA affinity chromatography and size exclusion chromatography following previously described protocols.

RAD-TGT Synthesis: TGTs were designed using previously characterized DNA sequences with a 5′ extension to allow for HUH binding. The following oligonucleotides were purchased from Integrated DNA Technologies. To prepare TGTs, anchor and ligand strands were mixed in a 1.1:1 ratio and then annealed in a 1× annealing buffer by heating at 98° C. for five minutes followed by cooling at room temperature for 1 hour. Excess bottom strand was used to mitigate any single stranded fluorescent top strand which may be internalized resulting in false positives. RAD-TGTs were then generated by reacting HUH-ligand of interest with the annealed duplex in a 2:1 ratio. Reactions were performed in reaction buffer over 30 minutes at 37° C.

Surface Preparation: 96-well glass plates were incubated with 80 μL of 100 g/ml BSA-Biotin in PBS for 2 hours at room temperature. In specified contexts, 18.75 μg/ml of fibronectin was added to the BSA-biotin solution. Wells were rinsed 2× with cold PBS and incubated with 100 μg/ml neutravidin for 30 minutes at room temperature. Wells were rinsed once more and incubated with 80 μL of 1 μM RAD-TGT and were incubated at 4° C. overnight. For all experiments, volume in the well never fell below 50 μL.

Cell lines: U251 talin-KO and U251 CD-44 KO cells were prepared using published methods. TLN1 KO and CD44 KO were achieved using the CRISPR/Cas9 system. A guide RNA (sequence AACUGUGAAGACGAUCAUGG, SEQ ID NO:8) was created to target TLN1. A guide RNA (gRNA) was created to target exon 2 in CD44 human cell lines (GAATACACCTGCAAAGCGGC, SEQ ID NO:9). A co-transposition method was used to enhance screening for knockout clones. Briefly, cells were transfected with Cas9 nuclease, gRNA, PiggyBac transposases, and PiggyBac transposon plasmid containing puromycin selection using FuGENE following the manufacturer's protocol. Puromycin selection was performed, and single cell clones were generated using serial dilution. After transfections, the cells were split into single clones and western blot was used to confirm the knockout. WB also verified that there was not over expression of Talin2 in response to KO of Talin1.

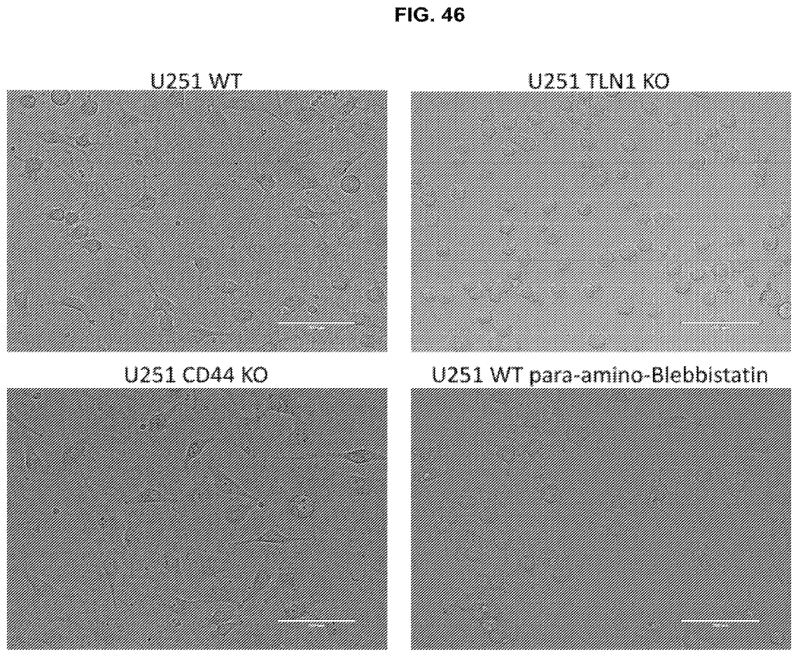

Cell Culture: Cells were maintained for no more than 15 passages. All cells were regularly passaged every 2 to 3 days when 80% confluency was achieved. All U251 and U251 knockout cells were maintained in Dulbecco's Modified Eagle Medium supplemented with 10% FBS and penicillin/streptomycin. CHO-K1 cells were maintained in F-12K medium supplemented with 10% FBS and penicillin/streptomycin. Brightfield images of cells cultured in conditions described in this Example are shown in FIG. 46.

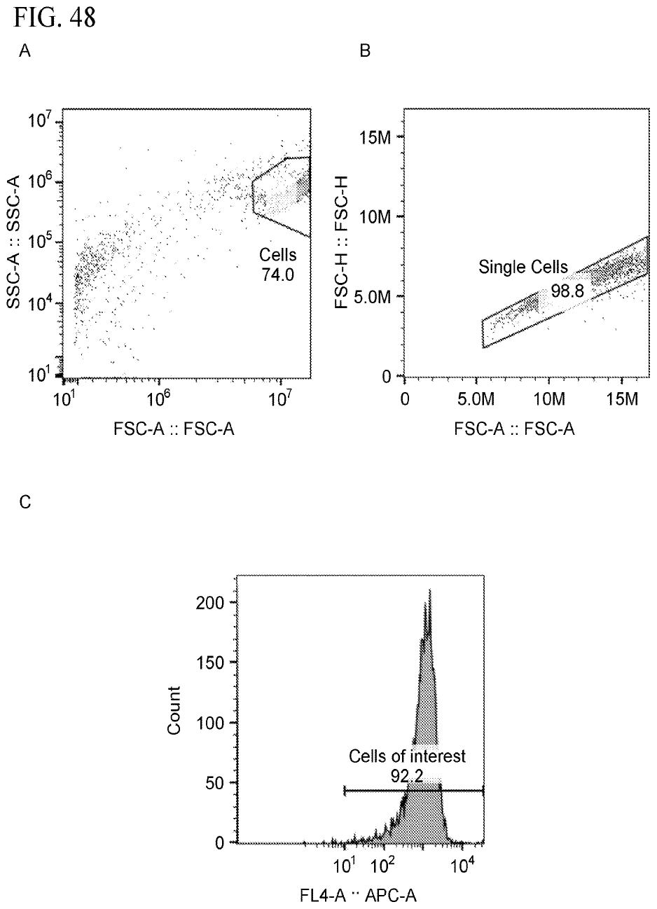

RAD-TGT Experimental Setup: Cells of interest were trypsinized for five minutes and transferred to Opti-MEM Reduced Serum Media. Cells were then counted using an automated cell counter. If cells were to be treated with para-amino-blebbistatin, the cells were incubated with 50 μM para-amino-blebbistatin at 37° C. for 30 minutes before application to the RAD-TGT surface. All para-amino-blebbistatin experiments were accompanied by a vehicle control consisting of cells being incubated in an equivalent volume of DMSO. RAD-TGT surfaces were washed once with cold PBS and 2× with Opti-MEM. Following washes and any drug treatment 15,000 cells were added to the wells and incubated at 37° C. with 5% CO₂ for desired period of time, typically 2 hours. Flow Cytometry Experiments: Following incubation on the RAD-TGT medium was removed and 30 μL of trypsin was added to each well. The plate was further incubated at 37° C. for five minutes. Following trypsinization 170 μL of flow cytometry buffer (PBS with 1% FBS and 1 mM EDTA) was added to the wells to quench the trypsin and resuspend the cells. Cells were removed from wells and analyzed with a BD ACCURI C6 Plus Personal Flow Cytometer without further washing. Cells were gated from the collected data, followed by gating for individual cells and then gated for cells that had a detectable signal (FIG. 48).

Microscopy: To visualize TGT ruptures surfaces were prepared following the above protocols. TGTs consisted of Quencher Ligand Strand and a Fluorescently Labeled Anchor Strand so if ruptured one should observe a gain of fluorescence on the glass surface. Rather than dissociating the cells post trypsinization, each well had the media removed and Fluorobrite DMEM was immediately added to wells. Cells were then imaged at 40× with an EVOS FL AUTO fluorescent microscope. Images were processed with ImageJ.

Statistical Analysis: In general, each experiment was performed on at least three separate days with 1-3 separated wells per day. The superplots were generated by importing histogram data from FlowJo into Prism. The ligand data were normalized to the median of the WDV-only data. The fluorescence data were concatenated to produce the dot plots. The medians of the normalized data for each biological replicate were used to overlay on the dot plot, and statistical significance was calculated using unpaired ANOVA as previously described.

### Example 2

This example describes that RAD-TGT signal is modulated by cytoskeletal inhibitors and loss of putative mechanosensors. It is shown that treatment with cytoskeletal modulating drugs such as para-amino-blebbistatin reproducibly reduced levels of measured fluorescence.

### Results

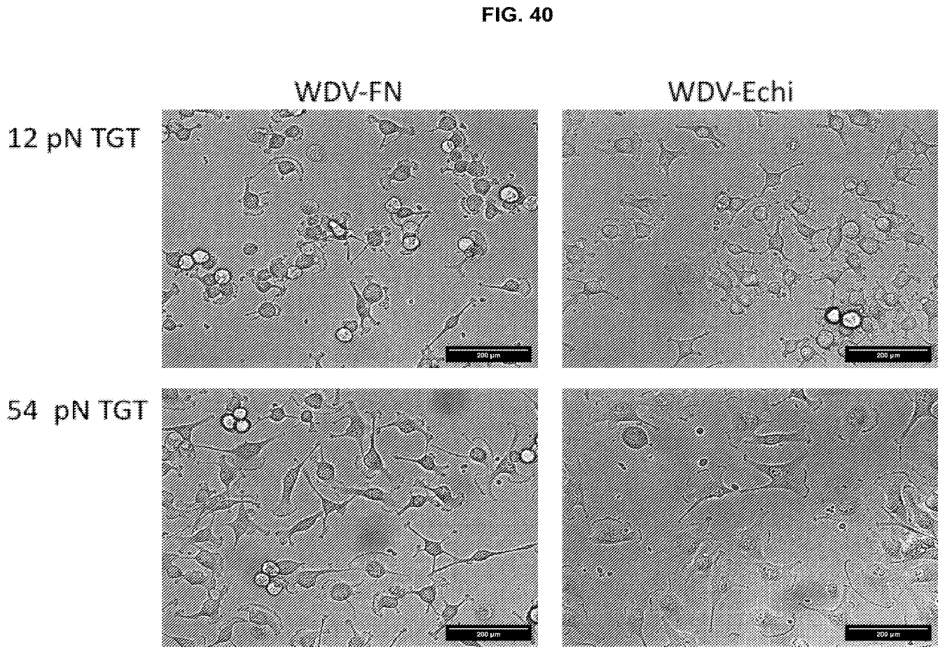

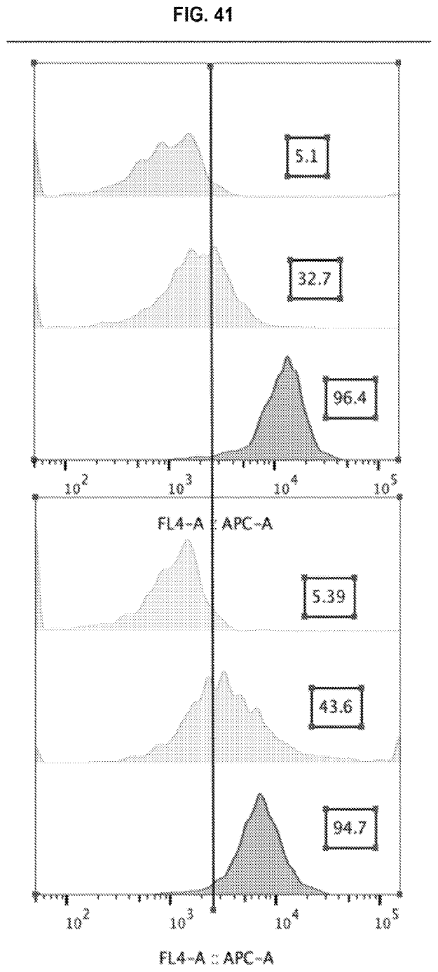

To gain more insight into the role of cellular forces on the observed fluorescence signal, the effects of cytoskeletal modulators on delivery of the ruptured oligo into the cell were measured. U251 cells were tested in addition to CHO-K1 cells. CHO-K1 cells and U251 cells did not adhere well to TGTs conjugated with WDV-FN (FIG. 20, FIG. 41). Brightfield images of U251 cells plated on different RAD-TGTs are shown in FIG. 40.

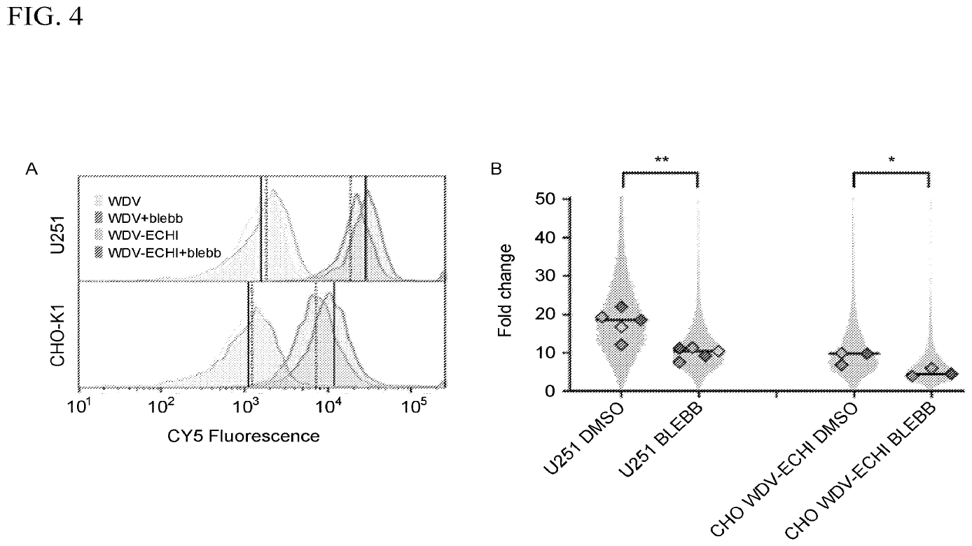

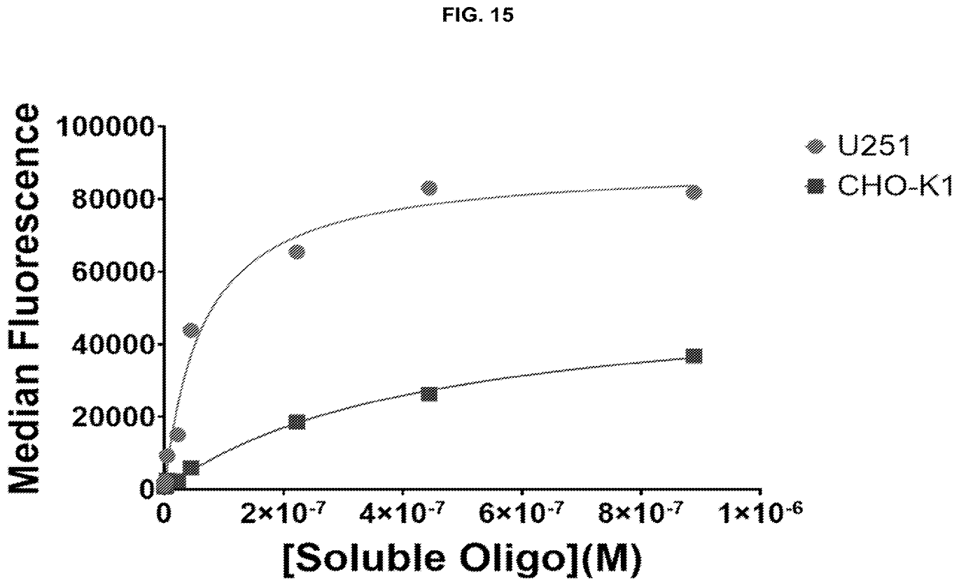

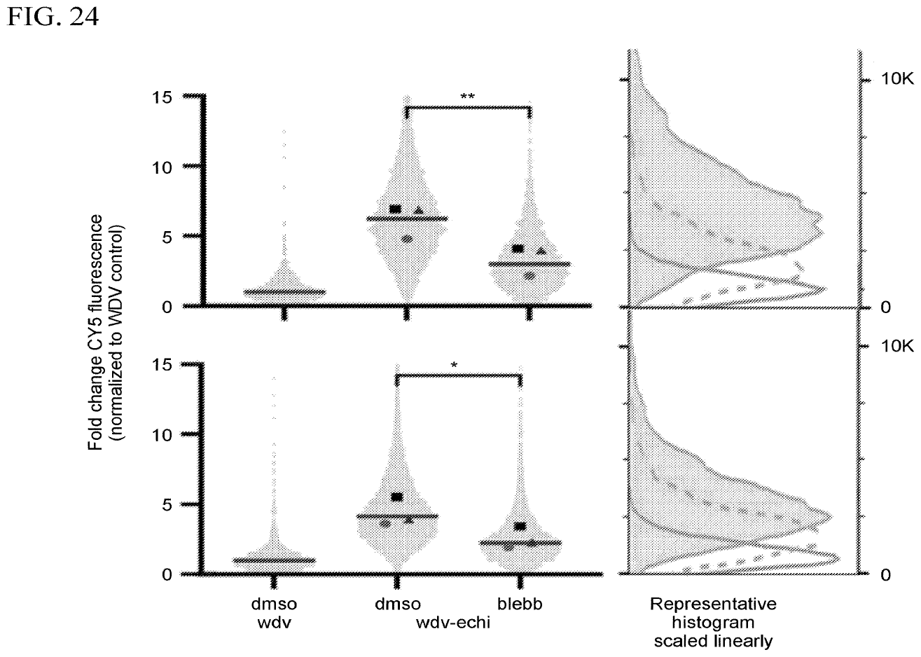

U251 cells exhibit an enhanced fold change in median fluorescence compared to CHO-K1 cells as shown in FIG. 4A-B and FIG. 24, suggesting they exert higher cellular forces. Indeed, an echistatin titration on U251 and CHO-K1 cells revealed a three-to-four-fold difference in bound echistatin, suggesting a greater number of integrin clutches available to exert traction force as shown in FIG. 15.

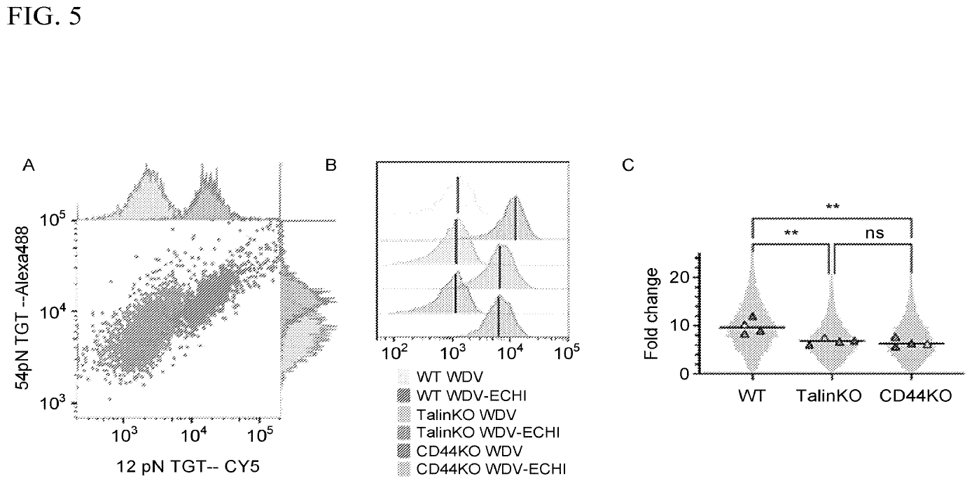

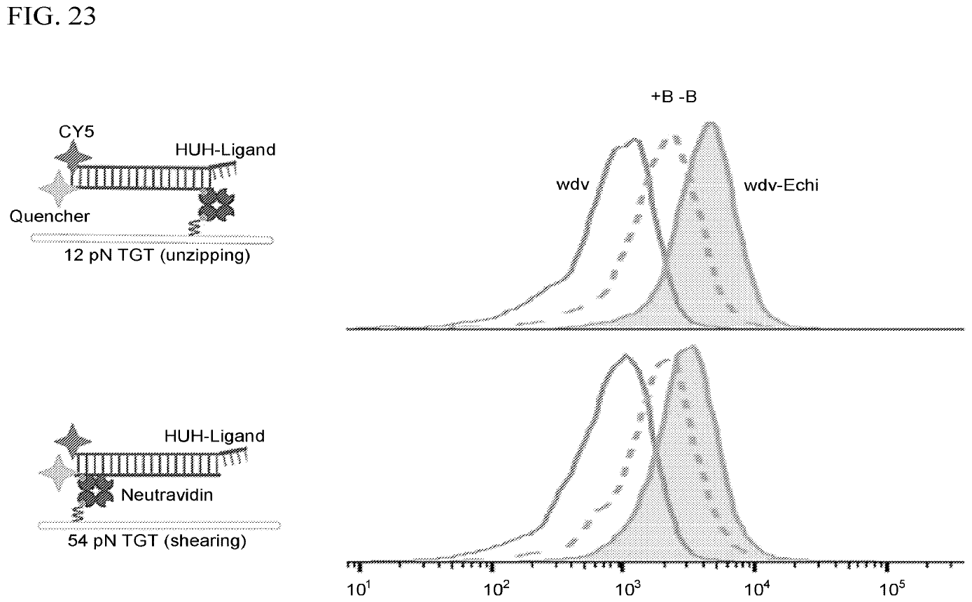

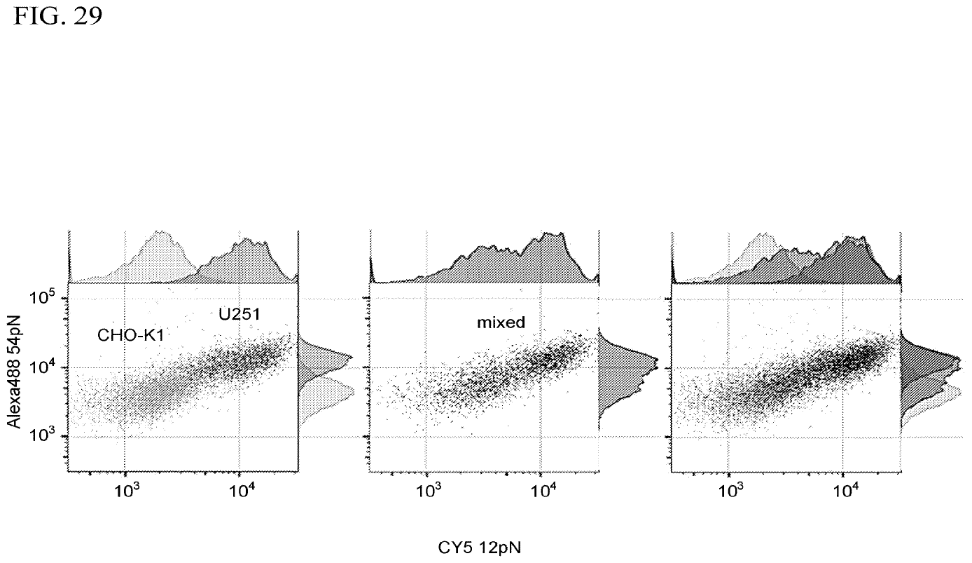

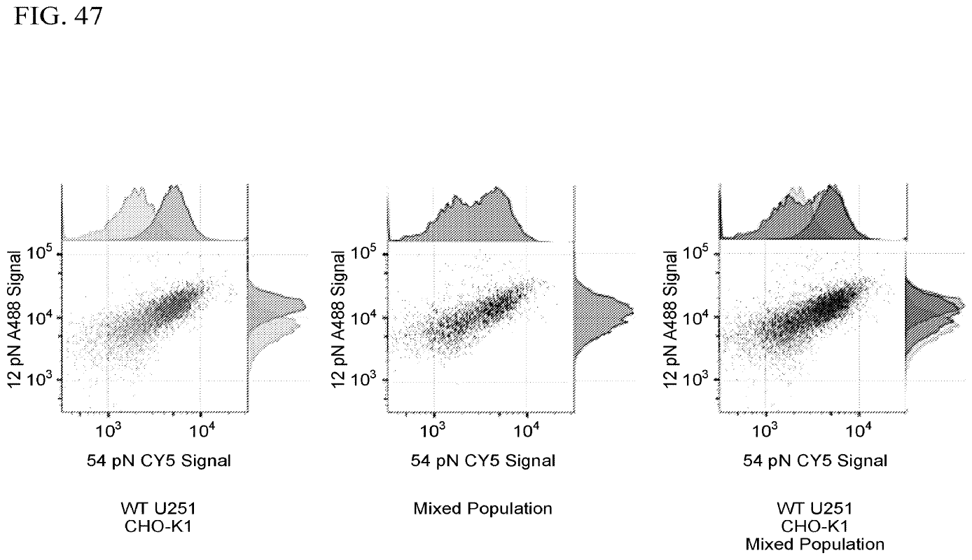

Next, CHO-K1 and U251 cells were plated on mixed RAD-TGTs with intrinsically different tension tolerances as shown in FIG. 5A. A mixture of CY5 labeled 12 pN anchor strand-ligand strand duplex and ALEXAFLUOR488-labeled 54 pN anchor strand-ligand strand duplex resulted in distinct populations, demonstrating that RAD-TGTs can be multiplexed to add additional parameters to the mechanical signature that could be useful in high throughput screening and flow sorting of cells exhibiting distinct mechanical signatures (FIG. 29, inverted results shown in FIG. 47). Generally, the 54 pN RAD-TGTs resulted in similar trends as the 12 pN tension tolerance (FIG. 23).

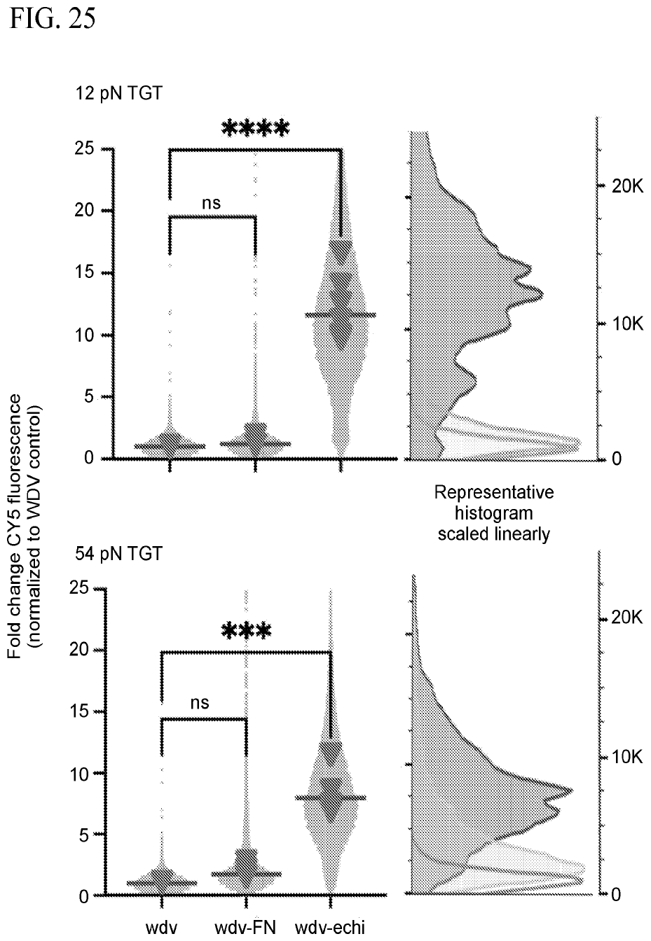

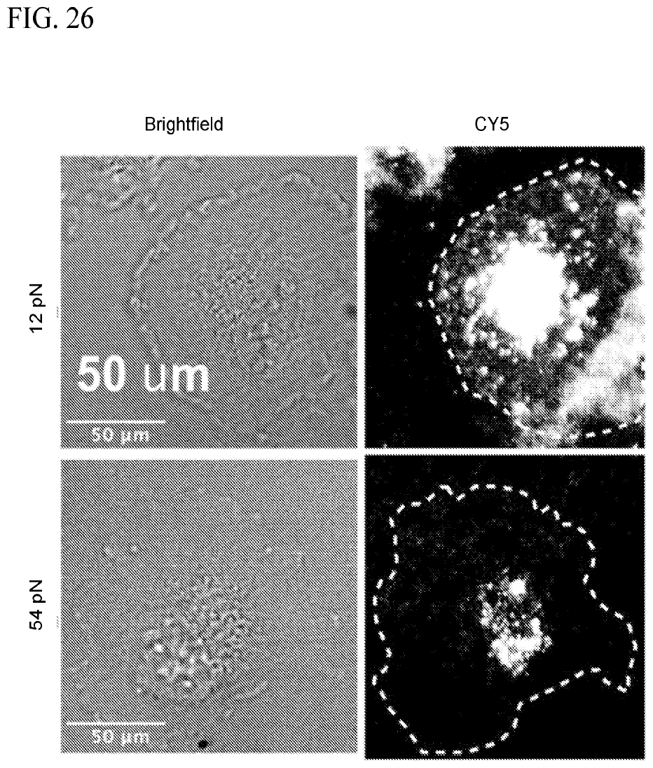

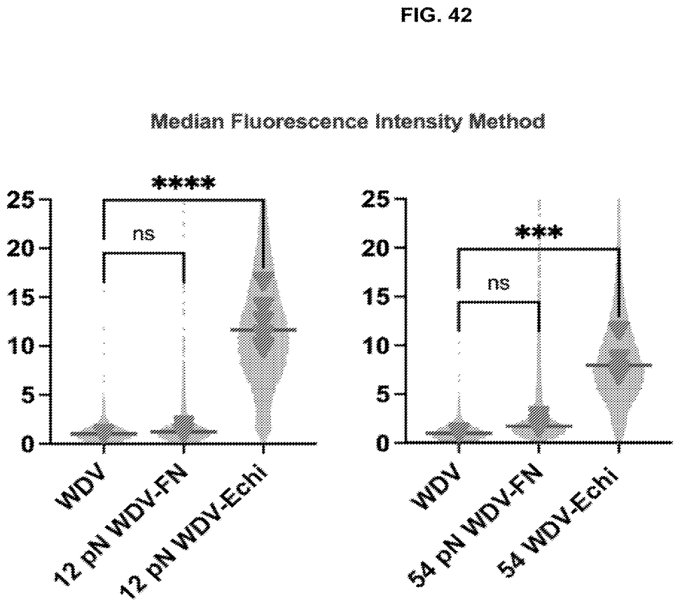

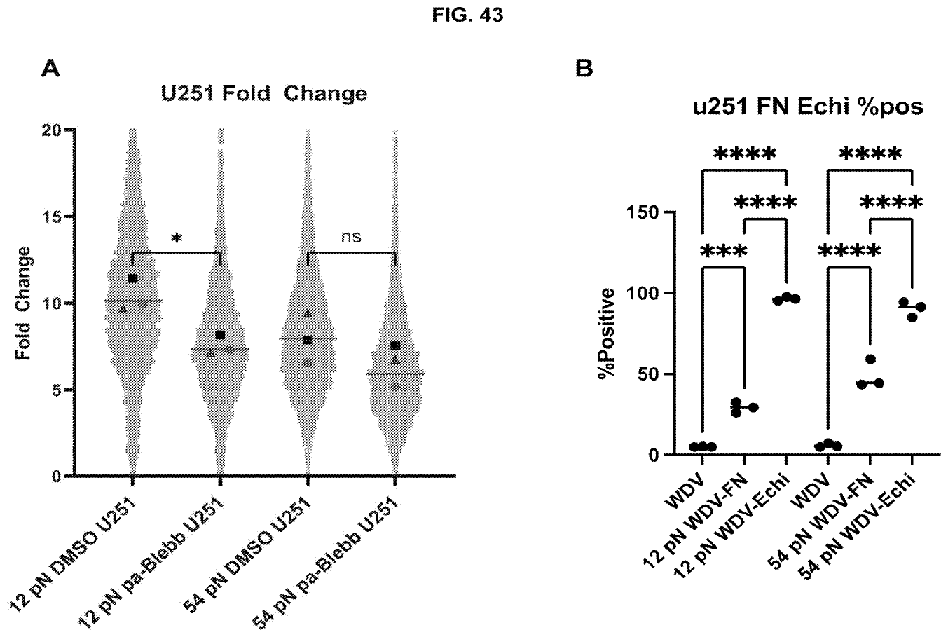

The fold change in median fluorescence was under twofold and not statistically significant, though the use of the median fluorescence to determine statistical significance is more stringent than other published methods which use the percentage of positive cells above an arbitrary gate (FIG. 41, FIG. 42, FIG. 43B). On WDV-Echi ligands, U251 cells generated signals much greater than CHO-K1 cells, with the 12 and 54 pN RAD-TGTs resulting in 12-fold and sevenfold increases in median fluorescence, respectively (FIG. 25, FIG. 26). Thus, U251 cells were used for experiments moving forward.

Next, cells were treated with myosin II inhibitor para-amino-blebbistatin as shown in FIG. 4, which has been shown to decrease TGT rupture in CHO-K1 cells and decrease traction force in U251 cells. Para-amino-blebbistatin treatment resulted in a consistent ~30% decrease in fluorescence in both cell lines as shown in FIG. 4B and FIG. 43A. These results suggest that the observed fluorescence signal is related to the traction force the cell is exerting.

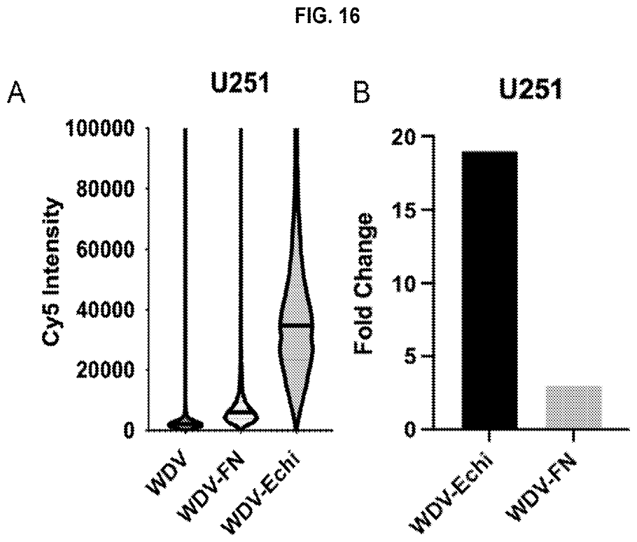

The high-throughput readout of RAD-TGT rupture could enable rapid screening for knockout of cellular proteins that alter cellular force generation. As proof of concept, wild type (WT) U251 cells were compared against U251 cells with CRISPR knockouts of two mechanosensing proteins: talin-1 and CD44, as shown in FIG. 5B and FIG. 5C. Talin-1 (TLN1) is a mechanosensor in the integrin-anchored focal adhesion complex while CD44 displays mechanosensitive behavior in glioma cells. U251 TLN1 KO cells displayed a rounded morphology on TGTs, while CD44 KO cells resembled wildtype U251 cells as shown in FIG. 16. Interestingly, RAD-TGT analysis of all three U251 cell lines (WT, TLN1 KO, CD44 KO) revealed statistically significant decreases in signal from both KO's relative to WT. These results suggest that knockouts of these genes decreased cellular force generation compared to wildtype cells, underscoring that the observed changes in traction force while also providing proof-of-concept for the use of RAD-TGTs in high throughput CRISPR screens.

### Experimental Methods

All experimental protocols were performed as described in Example 1.

### Example 3

This example describes that RAD-TGT can discriminate among ligands of different affinity

### Results

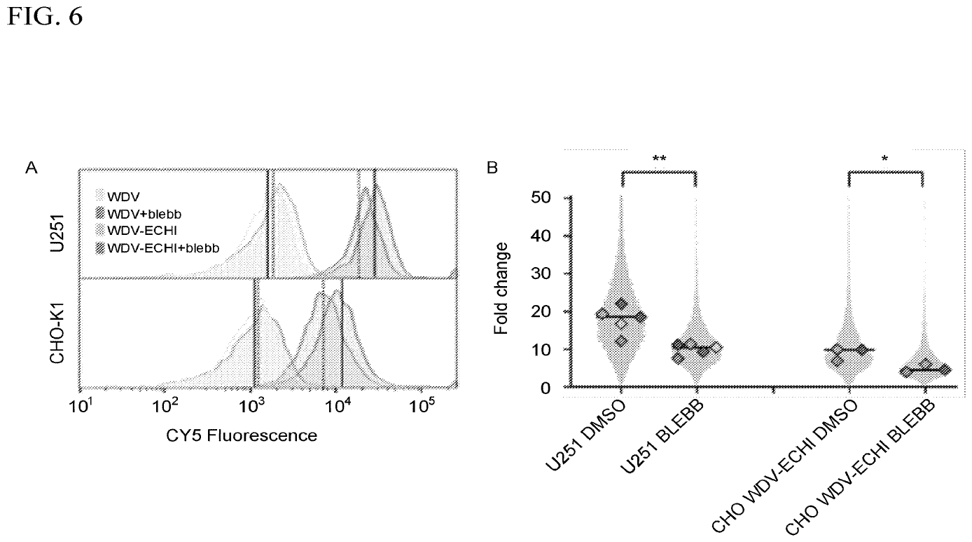

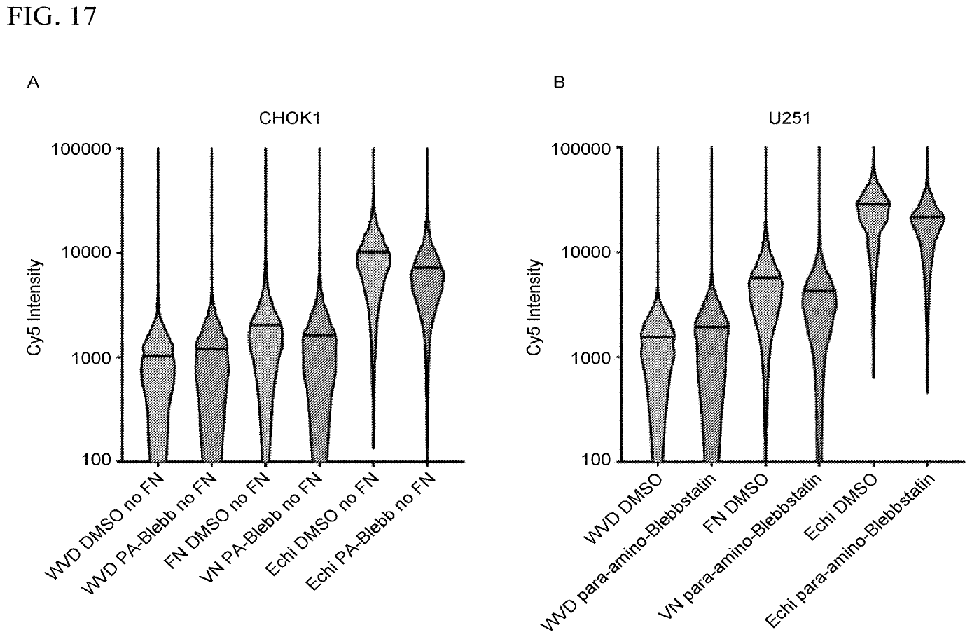

RAD-TGT readout was confirmed to be sensitive to the affinity of the ligand-receptor interaction, as the rupture of the DNA anchor strand-ligand strand duplex depends not only on the tension generated in a ligand-receptor complex but also on the kinetics of the ligand-receptor interaction. Echistatin is known to have a sub-nanomolar affinity for a broad range of integrin receptors, increasing the propensity to rupture TGTs. HUH fusions with a chimeric fibronectin domain (FN) were recombinantly expressed and purified as described herein. Strong differences in median fluorescence between the two ligands were observed, as shown in FIG. 6. These differences were detectable over background fluorescence as shown in FIG. 16 and FIG. 41. Though experiments with the FN ligand produced a lower fold change from the WDV negative control compared to echistatin, all of the trends observed with echistatin were recapitulated with the FN ligand as shown in FIG. 17.

### Experimental Methods

All experimental protocols were performed as described in Example 1.

### Example 4

This example describes that RAD-TGT rupture can be measured by DNA-sequencing. Three barcoded RAD-TGTs harboring different ligands were detected using Sanger sequencing and next-generation sequencing.

### Results

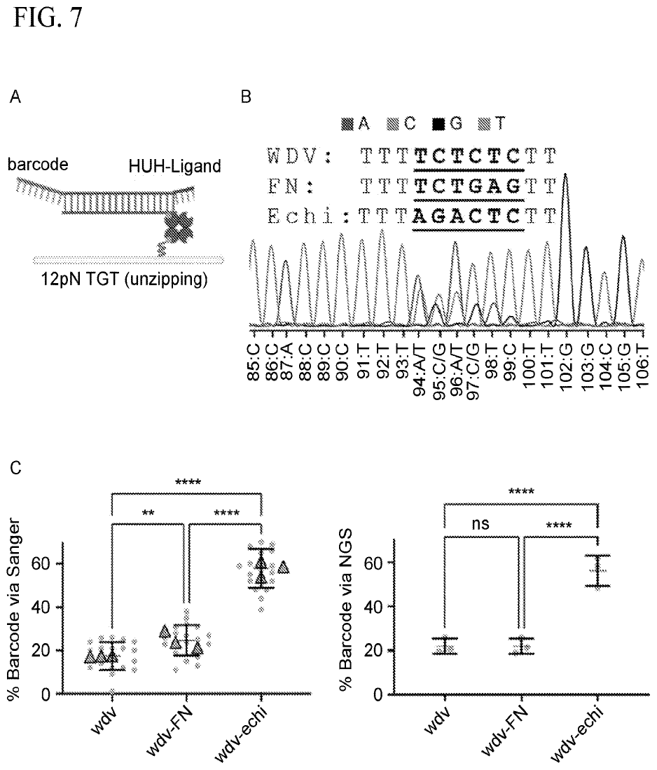

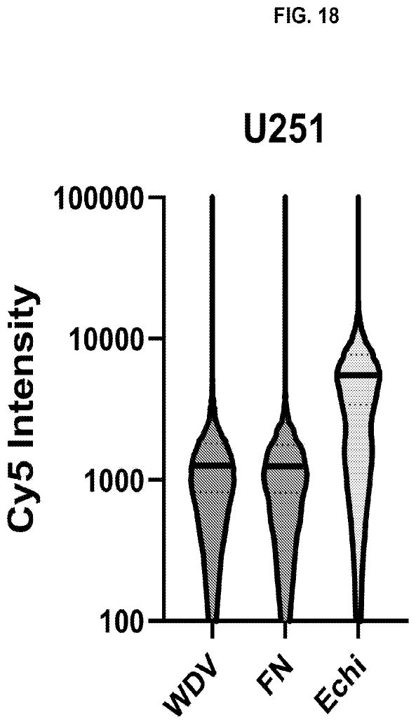

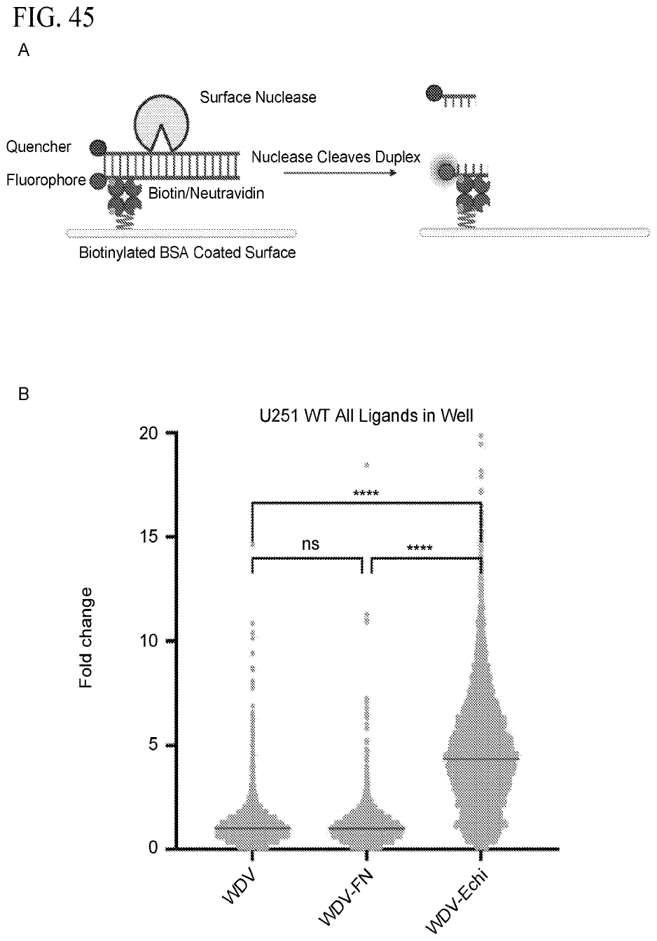

A barcode sequence was added to the 3′ end of the ligand strand of a population of RAD-TGT conjugated to HUH-Echi. A second barcode sequence was added to the 3′ end of the ligand strand of a population of RAD-TGTs conjugated to HUH-FN. A third barcode was added to the 3′ end of the ligand strand of a population of RAD-TGTs conjugated to HUH only. The sequences of the three barcoded ligand strands are shown in Table 2. All three barcoded RAD-TGTs were mixed and immobilized on the same surface. The barcode sequence was added to the 3′ end of the ligand strand. Cells were cultured on this surface as previously described. After a period of time cells were harvested, lysed, and barcodes were extracted and amplified. Amplified barcodes were sequenced using both Sanger sequencing or Illumina Next Generation Sequencing (NGS), as shown in FIG. 7A, FIG. 7B, and FIG. 7C. A web-based sequence deconvolution program was used to calculate the percent of each barcode represented in the samples quantified by Sanger sequencing. Biopython was used to calculate the percentage of each barcode represented in the samples quantified by NGS. A statistically significant increase in HUH-Echi relative to HUH and HUH-FN with both sequencing methods but a statistically significant increase in HUH-FN was observed only with Sanger sequencing. It should be noted that when the experiment was performed with fluorescent oligos and a flow cytometry readout the results mirrored the NGS data in which only echistatin was elevated as shown in FIG. 18. The robust echistatin signal relative to other ligands was attributed to the very strong affinity of echistatin for integrins. The discrepancies between Sanger sequencing and NGS were attributed to implicit error rates of sequencing that can arise from events such as PCR bias. Remarkably, the quantification of ruptured TGTs by sequencing corresponds with the trends seen in the analogous flow cytometry experiments (FIG. 45B), in which WDV-Echi provides a very robust signal relative to the co-plated ligands WDV and WDV-FN. This recapitulation of results via sequencing indicates that, indeed RAD-TGTs can be analyzed via sequencing.

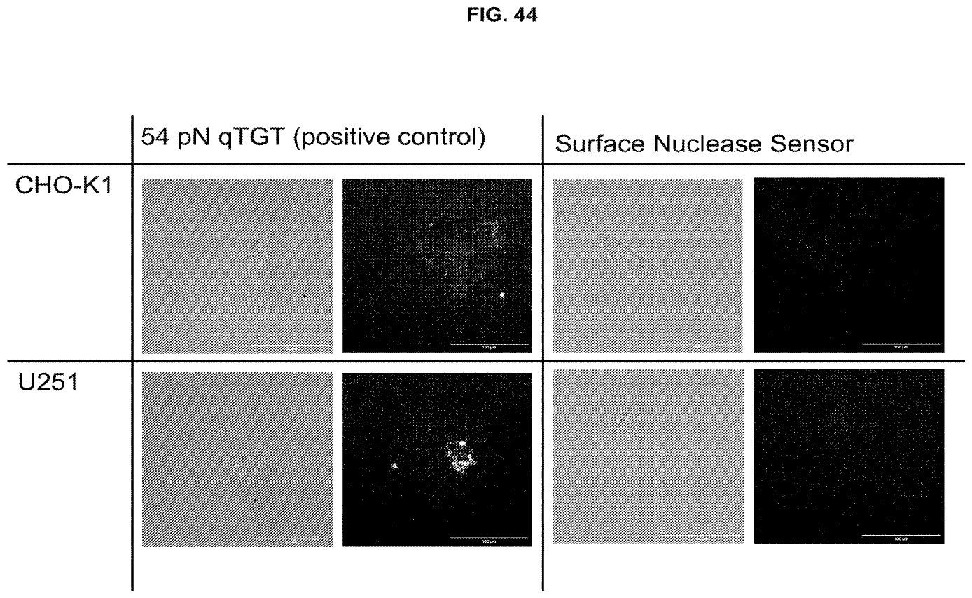

To ensure this increase in signal was not due to any differences in membrane-bound nuclease activity between cell lines, as has been observed for some cancer cell lines, a surface-bound nuclease sensor was used to visualize nuclease activity43. U251 cells exhibited very little nuclease activity compared to force-ruptured TGTs (FIG. 45A, FIG. 44). Furthermore, U251 cells had similar nuclease activity to CHOK1 cells which have previously been characterized as having low nuclease activity.

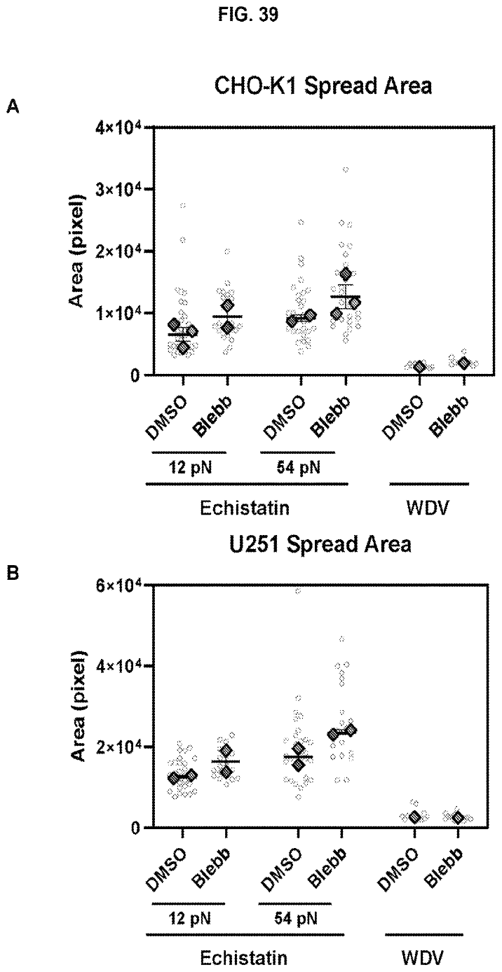

The decrease in signal following drug treatment indicates that the TGT rupture and subsequent internalization into the cell depend on cellular forces. Intriguingly, cell spread area slightly increased following treatment even as RAD-TGT signal decreased, which has been observed previously but indicates that changes in fluorescence as a result of rupturing RAD-TGTs is not simply a measure of cell spread area (FIG. 39).

### Experimental Methods

Sequencing Experiments: Sequencing experiments followed the same method as flow cytometry experiments, but cells were not analyzed with a cytometer. Three unique RAD-TGTs were in the experimental well, one for each ligand (WDV, FN, or Echi) and each RAD-TGT contained a novel barcode on the 3′ end of the ligand strand. Following removal from the well, cells were treated with 1× Passive Lysis Buffer for 20 minutes at room temperature while on an orbital shaker. The lysate had the barcoded top strands PCR amplified and the resulting PCR product was gel purified and submitted for Sanger and Illumina Sequencing. Quantification of barcodes in the Sanger sequences was performed using the Base-editing analysis software EditR. A dummy guide sequence was denoted that spanned the barcode area. Percentages at AGA and GAG in the barcodes were averaged to calculate percent of WDV-Echi and WDV-FN respectively for forward and reverse sequencings of three biological replicates. Prevalence of the WDV-only barcode was calculated from the difference from 100%. The program Tracy was used to visualize the sequence displayed in FIG. 3. Quantification of barcodes in the Next-Generation sequencing assay was performed with a custom Python script using the Biopython package. Briefly, forward sequencing reads were parsed, trimmed to include only the region containing the barcode, and barcodes were then counted. Reverse sequencing reads were reverse complemented and then processed in an identical manner. Forward and reverse counts for the three barcodes in each sample were combined and barcodes corresponding to either WDV-Echi, WDV-FN, or WDV only were exported for each sample.

### Example 5

Mechanotype is Altered when Putative Mechanosensing Proteins are Knocked Out.

The high-throughput mechanotype readout resulting from RAD-TGT rupture could enable CRISPR screening for the knockout of cellular proteins that alter cellular force generation. As proof of concept, the propensity of wildtype U251 cells (WT) to rupture RAD-TGTs was compared to U251 cells with CRISPR knockouts of two putative mechanosensing proteins-talin-1 and CD44 (FIG. 27A). Talin-1 is a mechanosensor in the integrin-anchored focal adhesion complex. Loss of talin-1 alters cell spreading and focal adhesion formation but does fully ablate activity due to the compensatory functions of talin-246. CD44 displays mechanosensitive behavior in glioma cells47 and is also thought to participate in crosstalk with integrin-based focal adhesions via common binding to the actin cytoskeleton. U251 talinKO cells displayed a rounded morphology on TGTs, while CD44 cells resembled wildtype U251 cells (FIG. 46). Knockout of both mechanosensing proteins reduced the median fluorescence upon rupture of 12 pN TGTs by 25-30% (FIG. 27A), with talin having a slightly larger effect on TGT rupture. Interestingly, differences in mechanical phenotype emerged when knockout cell lines were plated on 54 pN TGTs. Talin-KO also significantly decreased cellular force exerted on 54 pN TGTs (~35% decrease in fold change). However, the effect of knockout of CD44 was blunted on 54 pN RAD-TGTs (~15% decrease), revealing potentially interesting mechanistic differences in the roles that talin and CD44 play in force sensing and also revealing that multiplexing of TGTs with different tension tolerances can provide new mechanistic insights, as others have demonstrated.

### RAD-TGTs can Detect Multiple Cell Populations in 1D and 2D Histograms

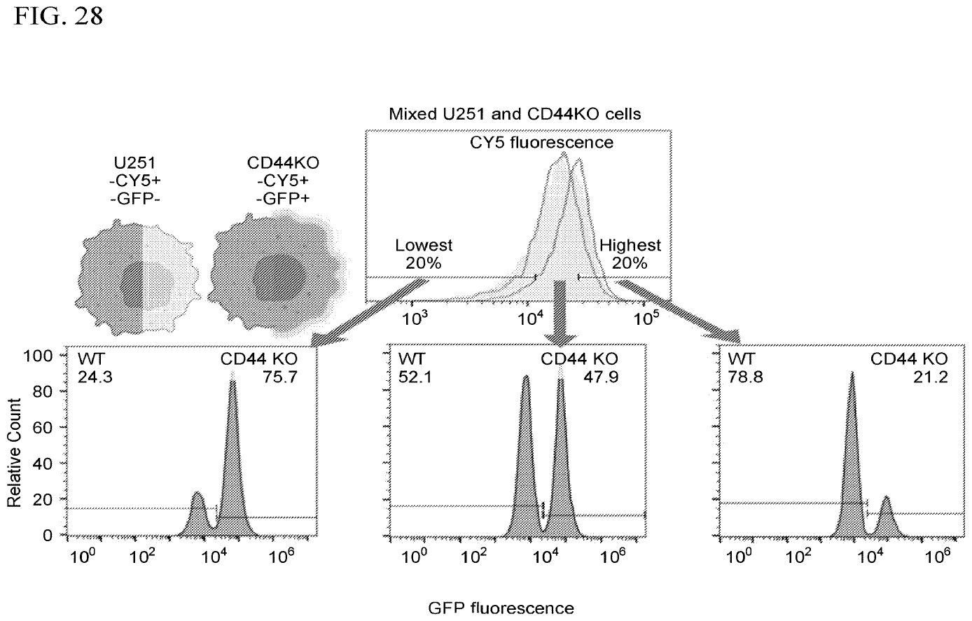

The RAD-TGT fluorescence histograms of the three cell lines resulted in distinct, separated peaks (FIG. 27B). A mixture of the three cell lines were plated and the fluorescence envelope resembled the sum of the three individual populations (FIG. 27B). Indeed, violin plots of the cumulative fluorescence plots of the three individual populations closely resemble the mixed cell population (FIG. 27C). In testing whether gating on cells at the high or low end of a mixed cell fluorescence profile would enrich for expected cells based on their mechanotype, CD44KO cell expression of a GFP transgene was exploited. WT and CD44KO cells were mixed and plated on 12 pN RAD-TGTs (FIG. 28). The resulting fluorescence profile of the mixed population (FIG. 28) recapitulates the two underlying cell populations, the cells were then gated on the highest and lowest 20% of cells on the edges of the mixed cell fluorescence profile and the GFP+ populations were observed. There were equivalent high and low GFP populations for the entire CY5 profile. The 20% of cells exhibiting the lowest CY5 fluorescence is enriched for high GFP, which corresponds to CD44KO cells. In contrast, the top 20% of fluorescent cells is enriched in the low GFP WTU251 cells. These inquiries demonstrate the potential of using RADTGTs in pooled CRISPR screens to sort cells with mechanotypes distinct from parent cells to identify genes that may contribute to that mechanotype. These results corroborate initial findings of CD44 KO exerting decreased forces on DNA tension probes compared to wildtype while also demonstrating that one can identify cells with altered mechanotype in a mixed population. Next, profiles of mixed populations of cells were measured on a mixture of 12 and 54 pN TGTs, resulting in 2D dot plots (FIG. 29) to potentially improve population separation and resolution. To do so, surfaces containing a 12 pNCy5 labeled RAD-TGT and a 54 pN Alexa488 RAD-TGT were prepared with the rationale that having two dimensions should increase the resolution and dimensionality of the mechanotype. This system was tested with WT U251 cells and CHO-K1 cells either individually or in the context of a mixed population. Much like the 1D plots, the mixed population of cells appeared as a composite of the two cell lines (FIG. 29). Furthermore, the experiment was repeated with fluorophores swapped between RAD-TGTs, and results were maintained (FIG. 47). The 2D plot serves as a promising next step in RAD-TGT development and usage.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A molecular tension sensor comprising:
a first nucleic acid strand nucleic acid;
a second nucleic acid strand nucleic acid, wherein the second nucleic acid strand comprises a region of at least five nucleotides that are complementary to a region of at least five nucleotides of the first nucleic acid strand; and
an HUH-tag.

2. The molecular tension sensor of claim 1, further comprising a ligand.

3. The molecular tension sensor of claim 2, wherein the ligand and the HUH-tag comprise a fusion protein.

4. (canceled)

5. The molecular tension sensor of claim 2, wherein the ligand comprises a nanobody, an antibody, or an scFv.

6. The molecular tension sensor of claim 1, wherein the first nucleic acid strand is immobilized to a surface.

7-9. (canceled)

10. The molecular tension sensor of claim 1, wherein the first nucleic acid strand is 5 nucleotides to 80 nucleotides in length.

11. The molecular tension sensor of claim 1, wherein the second nucleic acid strand is 20 nucleotides to 100 nucleotides in length.

12. The molecular tension sensor of claim 1, wherein the second nucleic acid strand comprises a fluorophore.

13. (canceled)

14. (canceled)

15. The molecular tension sensor of claim 1, wherein first nucleic acid strand comprises a fluorescence quencher.

16. The molecular tension sensor of claim 1, wherein the region of complementary nucleotides of the second nucleic acid strand is 10 nucleotides to 30 nucleotides in length.

17. (canceled)

18. (canceled)

19. The molecular tension sensor of claim 1, wherein the second nucleic acid strand comprises a barcode sequence.

20. (canceled)

21. The molecular tension sensor of claim 1, wherein the first nucleic acid strand, the second nucleic acid strand, or both comprises modified nucleotides.

22. A plate for analysis of molecular tension in cultured cells comprising the molecular tension sensor of claim 1.

23-28. (canceled)

29. A kit for analysis of molecular tension, the kit comprising the molecular tension sensor of claim 1, one or more buffers, and instructions for use.

30. A method of preparing a molecular tension sensor, the method comprising:
providing a first nucleic acid strand immobilized to a substrate;
providing a second nucleic acid strand, wherein the second nucleic acid strand comprises a region of nucleotides complementary to the first nucleic acid stand and a region of nucleotides recognizable by an HUH-tag;
hybridizing the second nucleic acid strand and the first nucleic acid strand, wherein hybridizing comprises annealing the region of complementary nucleotides to form a hybridized assembly; and
reacting the second nucleic acid strand with an HUH-tag, wherein the HUH-tag comprises a ligand.

31. A method of measuring molecular forces in a cell, the method comprising:
providing a surface comprising the molecular tension sensor of claim 1;
culturing a cell on the surface;
measuring the molecular forces in the cell, wherein measuring comprises measuring the presence of the molecular tension sensor; and
interpreting the measurement of the presence of the molecular tension sensor as it relates to molecular forces.

32. The method of claim 31, wherein measuring the presence of the molecular tension sensor comprises measuring fluorescence.

33. The method of claim 32, wherein fluorescence is measured using flow cytometry.

34. (canceled)

35. The method of claim 31, further comprising lysing the cell, extracting DNA from the cell, amplifying the DNA, and sequencing the DNA.

36-40. (canceled)

41. The molecular tension sensor of claim 1, wherein the first nucleic acid strand, the second nucleic acid strand, or both comprises one or more peptide nucleic acid nucleotides.

42. (canceled)


SEQUENCE LISTING (10 sequences)

SEQ ID NO: 1 - DNA, 45 bp, Synthetic construct
  source 1..45: mol_type = other DNA
gctataaactcaccgtaattttttggcccgcagcgaccacccttt

SEQ ID NO: 2 - DNA, 18 bp, Synthetic construct
  source 1..18: mol_type = other DNA
gggtggtcgctgcgggcc

SEQ ID NO: 3 - DNA, 71 bp, Synthetic construct
  source 1..71: mol_type = other DNA
gctataaactcaccgtaattttttggcccgcagcgaccacccttttctctcttggcgtcatcgtgtaccgg

SEQ ID NO: 4 - DNA, 71 bp, Synthetic construct
  source 1..71: mol_type = other DNA
gctataaactcaccgtaattttttggcccgcagcgaccaccctttagactcttggcgtcatcgtgtaccgg

SEQ ID NO: 5 - DNA, 71 bp, Synthetic construct
  source 1..71: mol_type = other DNA
gctataaactcaccgtaattttttggcccgcagcgaccacccttttctgagttggcgtcatcgtgtaccgg

SEQ ID NO: 6 - DNA, 122 bp, Synthetic construct
  source 1..122: mol_type = other DNA
acactctttccctacacgacgctcttccgatctctcaccatgtggtgacgccagaatttgccgcaatacacagtttacgccgttcggtcagcttggtatccgtagcgcagcgaccacccttt

SEQ ID NO: 7 - DNA, 120 bp, Synthetic construct
  source 1..120: mol_type = other DNA
gactggagttcagacgtgtgctcttccgatctgagaaacctaagcagacttctcctggtcgatgattgataagggtctcggaatgtccccggtcgcatggttccggtacacgatgacgcc

SEQ ID NO: 8 - RNA, 20 nt, Synthetic construct
  source 1..20: mol_type = other RNA
aactgtgaagacgatcatgg

SEQ ID NO: 9 - RNA, 20 nt, Synthetic construct
  source 1..20: mol_type = other RNA
gaatacacctgcaaagcggc

SEQ ID NO: 10 - DNA, 22 bp, Synthetic construct
  source 1..22: mol_type = other DNA
ccaccctttwswstcttggcgt